(19) United States
(12) Patent Application Publication
(10) Pub. No.: US 2011/0208162 A1
(43) Pub. Date: Aug. 25, 2011

(54) METHODS FOR PREVENTING AGGREGATION OF ADIPOSE STROMAL CELLS

(75) Inventors: Keith L. March, Carmel, IN (US); Brian H. Johnstone, Indianapolis, IN (US)

(73) Assignee: Indiana University Rsearch and Technology Corporation, Indianapolis, IN (US)

(21) Appl. No.: 13/125,563

(22) PCT Filed: Oct. 26, 2009
(86) PCT No.: PCT/US09/62120

§ 371 Date: Apr. 21, 2011

Related U.S. Application Data
(60) Provisional application No. 61/108,337, filed on Oct. 24, 2008.

(51) Int. Cl. A61K 35/12; C12N 5/074; C12N 5/10; A61P 9/10; A61P 9/00; A61M 25/00

(52) U.S. Cl. 604/522; 435/366; 424/93.7

(57) ABSTRACT

The present invention relates to a methods, compositions, and admixtures which prevent the aggregation of a population of adipose stromal cells (ASCs), such as an isolated population of adipose stromal cells. In some embodiments, the present invention relates to admixtures and methods of use thereof comprising a population of ASC and a modulator of ASC aggregation. In some embodiments, a modulator of ASC aggregation includes, for example, ionic agents (e.g., heparin), chelating agents (e.g., EDTA), proteolytic agents.(e.g., trypsin or dispase), and agents which inhibit the expression of cell surface receptors and molecules on the surface of ASCs (e.g., inhibitors of integrins expression). In some embodiments, the methods to block ASC aggregation and the admixtures are useful in use of the ASCs to treat various diseases and/or conditions, and increases the safety and/or efficiency of ASCs. The methods, compositions and admixtures have utility in treating a number of conditions including, but not limited to, the treatment of any vascularized organs, such as heart, kidney, liver and the like.

### CROSS REFERENCE TO RELATED APPLICATIONS

This application claims the benefit under 35 U.S.C. 119(e) of U.S. Provisional Patent Application Ser. No. 61/108,337 filed 24 Oct. 2008, the contents of which are incorporated herein by reference in their entirety.

### FIELD OF THE INVENTION

The present invention generally relates to regenerative cells derived from adipose tissue, and more particularly to adipose derived stromal cells (ASC), and method and compositions for preventing the aggregation of adipose derived stromal cells (ASC) for treatment, transplantation and regenerative medicine.

### BACKGROUND OF THE INVENTION

Adipose-derived stromal cells (ASC) limit injury from myocardial infarction. However, limited safety data are available for the transplantation of these cells. Particularly, the effects on normal myocardium have not yet been evaluated. Therefore data and methods are needed to improve the safety of ASC delivery.

Despite recent advances in the medical and interventional treatment of coronary artery disease, the loss of cardiac tissue and resulting impairment of the left ventricular function after a myocardial infarction (MI) remains the most common cause of heart failure. Recent pre-clinical as well as clinical trials involving a range of cell types, including bone marrow-derived mononuclear cells and skeletal myoblasts have suggested that stem or progenitor cell therapy will be a potential therapeutic modality for repair of damaged myocardium and attenuation of remodeling, thereby improving clinical outcomes.^{1,2} Among the various cell types, adipose tissue-derived stromal cells (ASC) have been shown to bear great similarity to bone marrow-derived mesenchymal stem cells (MSC) with regard to morphology, marker and immune phenotype, and differentiation capacity.^{3.4} In contrast to bone marrow, subcutaneous adipose tissue can be easily and safely harvested in large quantities, which have sufficient doses of ASC, and accordingly there is no need for further expansion in cultures. Based on this availability, rapidity of isolation, and characteristics overlapping with other mesenchymal stem cells, ASC are emerging as an attractive source of cells for cardiac repair.^{5,6} However, limited safety data are available, especially in the non-MI model. Such data are of particular interest given recent reports of coronary flow impairment following intracoronary administration of bone marrow-derived mesenchymal stem cells.⁷ The inventors demonstrate the safety and tolerability of an intracoronary injection of freshly isolated ASC in normal pigs. In particular, the inventors demonstrate administering (via intracoronary injection) a ASC in combination with an agent which inhibits aggregation of ASC.

### SUMMARY OF THE INVENTION

The invention is based upon the discovery that intracoronary delivery of ASC was shown to result in small infarctions accompanied by subacute, self-limited cVR. The inventors demonstrate that safety in patients can be increased by minimizing delivery to normal myocardium, and administering the ASCs in an admixture with heparin to minimize the effects of ASC cell aggregation.

Isolated adipose stromal cells (ASCs) were harvested from fat for reinjection into the coronary arteries of pig hearts and it was observed that these cells formed macromolecular aggregates. Microscopic inspection of isolated cells in solution indicated that cell aggregates ranged from 4 cells (the lowest number of cells in an aggregate that could be reliably counted) to numbers which were too numerous to count. This aggregation phenomenon was observed with ASCs isolated from porcine and human fat that were either freshly harvested or had been cultured. The inventors discovered the nature of the properties of ASCs that contributed to aggregation and demonstrate methods to block aggregation of ASC.

Accordingly, one aspect of the present invention relates to methods to inhibit cell aggregation, and in particular to inhibit cell aggregation of ASCs. In some embodiments the methods and compositions are useful inhibiting aggregation of ASCs, or other cell types with similar aggregative properties.

One embodiment is a method whereby the aggregative properties of isolated ASCs are inhibited by treatment with substances that block this process. Treating ASCs in this manner prevents the formation of macromolecular aggregates that may block arteries and thereby reduce blood flow to tissues; thus, enhancing the safety profile of ASCs when delivered as a therapeutic agent for treatment of diseases of vascularized tissues and organs, such as when used in the treatment of myocardial infarction.

The inventors have discovered methods and compostions to block aggregation ASCs using agents that inhibit aggregation, which can include ionic agents (e.g., heparin), chelating agents (e.g., EDTA), proteolytic agents.(e.g., trypsin or dispase), intracellularly acting agents that block expression of surface displayed attachment proteins (e.g., integins), or any other agent that modifies attachment or self-aggregative properties of ASCs.

Blocking the aggregation of ASCs using the methods and compositions as disclosed herein, especially before using ASCs to treat various diseases and/or conditions, increases the safety and/or efficiency of these cells. Treated ASCs have utility in treating a number of conditions including, but not limited to, the treatment of any vascularized organs, such as heart, kidney, liver and the like.

One aspect of the present invention relates to an admixture comprising a combination of an isolated population of adipose-derived stromal cells (ASCs) and at least one modulator of ASC aggregation. In some embodiments, the modulator of ASC aggregation inhibits aggregation of the ASCs.

Another aspect of the present invention relates to a method of inhibiting formation of macromolecular aggregates of adipose-derived stromal cells (ASC), comprising contacting a population of ASC with a modulator of ASC aggregation. In some embodiments, a macromolecular aggregate of ASC comprises at least 4 adipose-derived stromal cells.

In some embodiments, a modulator of ASC aggregation is an ionic agent, for example, heparin or an analogue thereof. In another embodiment, a modulator of ASC aggregation is a chelating agent, for example, EDTA, EGTA or an analogue thereof. In some embodiments, a modulator of ASC aggregation is a proteolytic agent, for example, trypsin or dispase or an analogue thereof. In some embodiments, a modulator of ASC aggregation is an agent which blocks the expression of an attachment protein expressed on the cell surface of ASCs, for example, an agent which blocks the expression of integrin, such as, but not limited to, a neutralizing antibody, peptide, protein, aptamer, ribosome, nucleic acid, RNAi, miRNA or small molecule inhibitor of an integrin expressed on the cell surface of an ASC.

In some embodiments, an admixture comprises human ASC, for example, ASC which are harvested from a human subject. In some embodiments, a population of ASCs are freshly isolated ASCs or in some embodiments, are cultured ASC. In some embodiments, a population of ASC comprise at least one genetically modified ASC.

In some embodiments, an admixture as disclosed herein is cryopreserved.

Another aspect of the present invention relates to the use of the admixture as disclosed herein for administering to a subject in need of a transplant of adipose-derived stromal cells (ASC). In some embodiments, the subject is affected with insufficient cardiac function or disease or disorder, for example, heart failure or myocardial infarction.

In some embodiments, an admixture as disclosed herein can comprises one or more additional agents, or any combination thereof selected from the group consisting of: angiogenic factors, growth factors, and immunosuppressive drugs.

In some embodiments, an admixture as disclosed herein can be administered to a subject via any suitable route known to one of ordinary skill in the art, for example, but not limited to administration is via endomyocardial, epimyocardial, intraventricular, intracoronary, retrograde coronary sinus, intra-arterial, intra-pericardial or intravenous administration. In some embodiments, an admixture is administered to a subject with, or at risk of myocardial infarction or heart attack via intracoronary delivery.

In some embodiments, an admixture as disclosed herein is administered to a human subject. In some embodiments, a population of ASCs in an admixture as disclosed herein are autologous, i.e. harvested from the same subject to which the admixture is administered to. In another embodiment, the ASC are heterologous, typically from a tissue type matched donor.

Another aspect of the present invention relates to the use of heparin for inhibiting aggregation of freshly isolated ASC. In some embodiments, even if a subject has previously been administered heparin for use as a blood thinner, heparin can co-administered to subject which is also administered ASC or transplantation of a population of ASCs.

Another aspect of the present invention relates to a method of reducing the consequences of a myocardial infarction in a subject, the method comprising the steps of: (a) obtaining freshly isolating ASC from the subject; (b) forming a mixture of the freshly isolated ASC and heparin; and (c) delivering via intracoronary catheter the mixture to post-infarction myocardium of the subject; wherein the delivery of the mixture of freshly isolated ASC and heparin reduces the consequences of a myocardial infarction in the subject.

### BRIEF DESCRIPTION OF DRAWINGS

FIGS. 1A-1F show examples of photomicrographs of ASC preparation after resuspension in delivery medium without heparin. FIG. 1A shows afresh preparation without evident aggregation (20×); FIG. 1B shows a preparation revealing microaggregates 40 minutes after resuspension (20×); FIGS. 1C and 1D show preparations revealing macroaggregates 40 and 50 minutes after resuspension (4×). FIGS. 1E and 1F show a comparison of preparation of ASC(E) and ASC-H(F) 60 minutes following.

FIGS. 2A-2C shows results from coronary contrast motion. FIG. 2A shows results from coronary contrast motion in terms of TIMI flow, FIG. 2B shows TIMI framecounts, and FIG. 2C shows ejection fraction derived from ventriculography prior to and following cell or vehicle delivery. NS; not significant.

FIGS. 3A-3C show changes of regional wall thickness indices obtained from left ventriculography in the presence of ASC alone (FIG. 3B) or ASC and heparin (FIG. 3C) following cell or vehicle delivery (FIG. 3A). The left ventriculogram in the right lateral projection is divided into five regions, which are the anterobasal (AB) segment, anterolateral (AL) segment, apical (AP) segment, diaphragmatic (DI) segment and posterobasal (PB) segment. NS; nonspecific.

FIGS. 4A-4C show changes in cardiac troponin following administration of the combination of ASC and heparin. FIG. 4A shows the changes of cardiac troponin I following cell or vehicle delivery. 24 hour cTn-I is elevated in ASC and ASC-H groups. FIG. 4B shows a longitudinal view with a small focal area of minimal myocardial infarction composed of proliferating fibroblasts. The infarct comprised about 45% of the field. FIG. 4C shows a cross sectional view with a similar micro-infarct with proliferating fibroblasts and the lesion comprised about 30% of the image. Bar=50 microns.

FIGS. 5A-5B show quantitation of nerve sprouting in sections of myocardium. FIG. 5A shows a comparison of quantified analysis of GAP 43 immunostaining from control, ASC and ASC and heparin groups. FIG. 5B shows a comparison of quantified analysis of TH immunostaining in control, ASC and ASC plus heparin treated groups.

FIGS. 6A-6E show continuous ECG tracing with modified lead I. FIG. 6A shows nonsustained wide QRS rhythm with cycle length of 580 msec. The behavior looks like accelerated idioventricular rhythm. FIG. 6B shows a sustained Fast VT with cycle length of 342 msec. Bradycardia episodes. FIG. 6C shows 2:1 AV block, FIG. 6D shows a high degree AV block, FIG. 6E shows a Wenckebach AV block with escape ventricular beat.

FIG. 7 shows the time-dependent burden of the ventricular rhythm is exhibited as the percentage of the number of QRS complexes of a ventricular origin out of the total number of QRS complexes in a given duration of hours.

FIGS. 8A-8D show the characteristics of a consecutive ventricular rhythm(cVR) in 21 hours after cell delivery. FIG. 8A shows an average cycle length of ASC and ASC-H Pigs (P=0.00123), FIG. 8B shows an average median cVR duration in ASC and ASC-H Pigs (P═NS), FIG. 8C shows an average maximum duration of cycle length in ASC and ASC-H Pigs, (P=0.0318), FIG. 8D shows the % duration of VT and AIVR rhythm with respect to total analysis period (21 h post cell delivery) for ASC (#1, #5, #6, #7, #8) and ASC-H (#12 and #13). Each animal number can be identified in Table 1, as disclosed herein. Pigs #10 and #11 exhibit no cVRs in the 21 h post cell delivery and were omitted from this analysis. Each animal number can be identified in Table 1.

FIGS. 9A-9E show images of aggregation by Adipose-derived stem cells (ADSCs) and SVF (also referred to as adipose-derived stromal cells (ASCs). FIG. 9A shows a photograph of SVF suspended at a concentration of 2×10⁶ SVFs/ml at 20 minutes post-isolation, a typical amount of time that passes between cell isolation and administration into porcine hearts. FIG. 9B shows a micrographic image (200×) of individual cells of 10 mm average diameter. FIGS. 9A-9E show aggregates of varying sizes observed after 20 minutes in suspension.

FIG. 10 shows that heparin inhibits cell aggregation. Cells were resuspended in buffer in triplicate. To one dose Heparin (400 μg/ml) was added. The suspensions were incubated with vigorous agitation and at the indicated timepoints an aliquot was removed to quantitate the number of individual cells and aggregates.

FIG. 11 shows heparin-mediated inhibition of aggregation is dose dependent. EDTA detached ASCs were resuspended in buffer only or with the indicated concentrations of Heparin. After 2 hours of vigorous shaking the number of aggregates (>4 cells) and individual cells was quantitated. Values are mean±sem.

FIG. 12 shows that aggregation of ASCs is blocked by pretreating with trypsin or adding EDTA to the buffer during incubation. Cultured ASCs were detached from plastic using 2 mM EDTA. Detached cells were resuspended in triplicate in buffer and subjected to the following treatments: (1) none, (2) trypsin for 2 minutes for suspension (3) EDTA, (4) EDTA then 7 mM Ca²⁺, or (5) pretreating with neutralizing tissue factor (TF) antibody for 30 minutes. Values are mean±sem.

FIG. 13 shows quantitation of the effects of Heparin concentration on aggregate sizes. Cell suspensions were photomicrographed and individual or aggregated cells were quantitated with Image J software. Parameters were set to distinguish between single cells, aggregates of 4-10 cells (“small”) and aggregates comprised of >10 cells (“large”). Each bar represents the means of three separate samples with counts obtained from 3 fields.

### DETAILED DESCRIPTION OF THE INVENTION

The present invention generally relates to methods and compositions and admixtures to prevent aggregation of ASCs and to prevent the formation of macromolecular aggregates of adipose-derived stromal cells (ASCs). The methods improve the safety of ASC therapy, particularly when used in connection with delivery of ASC to vascularized organs or vasculature. The methods improve the safety of ASC therapy, particularly when used in connection with delivery of a population of ASC to the myocardium or for the treatment of cardiovascular disease disorders, such as heart failure or myocardial infarction.

Prevention of aggregation of ASC is important given that there is a high risk of coronary flow impairment e.g. following intracoronary administration of bone marrow-derived mesenchymal stem cells.⁷ Similarly, the inventors have demonstrated that intracoronary delivery of freshly isolated ASC results in small infarctions accompanied by subacute, self-limited cVR. The inventors also demonstrated that ASC can be safely used if delivery of ASCs to the normal myocardium is minimized, and that the ASC cells are targets to post-ischemic tissue and/or the ASCs are administered in an admixture with at least one agent which inhibits ASC aggregation, such as heparin. Accordingly, the inventors demonstrate the safety and tolerability of an intracoronary injection of freshly isolated ASC by administering to the subject an admixture comprising a population of ASCs in combination with at least one agent which inhibits aggregation of ASC.

The inventors demonstrate that isolated ASC harvested from fat for reinjection into the coronary arteries of pig hearts formed macromolecular aggregates. Microscopic inspection of isolated ASC from porcine and human fat in solution indicated that cell aggregates ranged from at least 4 cells to numbers which were too numerous to count.

Accordingly, one aspect of the present invention relates to methods to inhibit cell aggregation, and in particular to inhibit cell aggregation of ASCs. In some embodiments the methods and compositions are useful inhibiting aggregation of ASCs, or other cell types with similar aggregative properties.

One embodiment is a method whereby the aggregative properties of isolated ASCs are inhibited by treatment with substances that block this process. Treating ASCs in this manner prevents the formation of macromolecular aggregates that may block arteries and thereby reduce blood flow to tissues causing a safety concern for ASC transplantation. The methods thus enhance the safety profile of ASCs when delivered as a therapeutic agent for treatment of diseases of vascularized tissues and organs.

In one embodiment, ASC can be used in an admixture with one or more, or any combination of the following agents which block aggregation ASCs, such as, but not limited to ionic agents (e.g., heparin), chelating agents (e.g., EDTA), proteolytic agents.(e.g., trypsin or dispase), intracellularly acting agents that block expression of surface displayed attachment proteins (e.g., integins), or any other agent that modifies attachment or self-aggregative properties of ASCs.

The admixture comprising a population of ASC and an agent which blocks the aggregation of the ASCs is useful in the therapeutic use of ASC for transplant purposes, for example, for using ASCs to treat various diseases and/or conditions, increases the safety and/or efficiency of these cells. In some embodiments, an admixture of ASC and an inhibitor of aggregation is useful in treating a number of conditions including, but not limited to, the treatment of any vascularized organs, such as heart, kidney, liver and the like. In some embodiments, an admixture of ASC and a modulator of ASC aggregation (i.e. an inhibitor of ASC aggregation) is useful in treating a number of cardiac conditions or diseases including, but not limited to, heart failure and myocardial infarction.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It is to be understood that this invention is not limited to the particular methodology, protocols, cell lines, constructs, and reagents described as such. It is also to be understood that the terminology used herein is for the purpose of describing particular embodiments only, and is not intended to limit the scope of the present invention which will be limited only by the appended claims.

The term “adipose derived stromal cells” or ASCs” are used interchangeably herein with “stromal vascular fraction cells” or “SVFs” and refer to adult cells that originate from adipose tissue. A population of adipose derived stromal cells is a heterologous population of cells comprising at least one or at least 2 or the following population of cells; endothelial cells, mesenchymal stem cells, fibroblasts, smooth muscle cells, pericytes and adipose-derived stem cells, as well as additional other cell types not listed. In some embodiments, adipose derived stromal cells refers to a substantially pure population of adipose-derived stem cells. In some embodiments, adipose derived stromal cells does not refers to adipose derived regenerative cells. Adipose derived stromal cells (ASCs) can be easily harvested from adipose tissue and are substantially free of adipocytes and red blood cells and clonal populations of connective tissue stem cells. The adipose derived stromal cells are substantially devoid of cells, which includes extracellular matrix material from adipose tissue. Typically, the cells are isolated using methods as disclosed herein.

The term “adipose” as used herein refers to any fat tissue from a subject. The terms “adipose” and “adipose tissue” are used interchangeably. The adipose tissue may be brown fat, white fat or yellow fat or white adipose tissue, derived from subcutaneous, omental/visceral, mammary, gonadal, or other adipose tissue site. The adipose tissue has adipocytes and stroma. Adipose tissue is found throughout the body of an animal. For example, in mammals, adipose tissue is present in the omentum, bone marrow, subcutaneous space and surrounding most organs. Preferably, the adipose is subcutaneous white adipose tissue. The adipose tissue may be from any organism having fat tissue. Preferably, the adipose tissue is mammalian, most preferably, the adipose tissue is human. A convenient source of adipose tissue is from liposuction surgery, however, the source of adipose tissue or the method of isolation of adipose tissue is not critical to the invention. The adipose tissue may be autologous or heterologous to the transplant recipient.

The term “aggregate” is a collection or assembly of more than one cell in a group. An aggregate may comprise one or more different cell types (e.g. endothelial cells, mesenchymal stem cells, fibroblasts, smooth muscle cells, pericytes and adipose-derived stem cells) which are present within a population of ASCs.

The term “microaggregrate” refers to a microscopic collection or clump of 4 or more individual cells, e.g. a microscopic collection or clump of more than 4 individual adipose-derived stromal cells. A microaggregate may comprise one or more different cell types (e.g. endothelial cells, mesenchymal stem cells, fibroblasts, smooth muscle cells, pericytes and adipose-derived stem cells) which are present within a population of ASCs.

The term “modulator of ASC aggregation” refers to an agent which increases or decreases by a statistically significant amount the formation of, or amount (e.g. number) of ASC aggregates or ASC microaggregates in a population of ASCs. In some embodiments, a modulator of ASC aggregation is an agent which reduces by a statistically significant amount the formation of, or amount (e.g. number) of ASC aggregates or ASC microaggregates in a population of ASCs as compared to in the absence of the agent.

The term “tissue” as used herein is a broad term that is applied to any group of cells that perform specific functions, and includes in some instances whole organs (e.g. parathyroid) and/or part of organs, such as pancreatic islets. A tissue need not form a layer, and thus encompasses a wide range of tissue including bone marrow, skin, connective tissue (e.g. cells that make up fibers in the framework supporting other body tissues); and hematopoietic and lymphoid tissue (e.g. cells which function as part of the body's immune system that helps protect it from bacteria and other foreign entities).

The term “cell transplant” as used herein refers to a population of ASCs or ASCs in a cell mass for transplantation into a subject. A cell transplant of ASCs can comprise genetically modified ASCs, as well as ASCs which have been differentiated from other cells, such as stem cells, progenitors, iPS cells and the like. The population of ASCs which make up a cell transplant are referred to as “ASC transplant cells”.

The term “adult” as used herein, is meant to refer to any non-embryonic or non-postnatal juvenile animal or subject. For example the term “adult adipose-derived stromal cell,” refers to an adipose-derived stromal cell, other than that obtained from an embryo.

The term “graft” as used herein refers to the process whereby a free (unattached) cell, tissue, or organ integrates into a tissue following transplantation into a subject.

The term “allograft” refers to a transplanted cell, tissue, or organ derived from a different animal of the same species.

The term “xenograft” or “xenotransplant” as used herein refers to a transplanted cell, tissue, or organ derived from an animal of a different species. In some embodiments, a xenograft is a surgical graft of tissue from one species to an unlike species, genus or family. By way of an example, a graft from a baboon to a human is a xenograft.

The term “xenotransplantation” refers to the process of transplantation of living cells, tissues or organs from one species to another, such as from pigs to humans.

The terms “contacting” or “contact” as used herein in connection with contact with an ASC, either present on a scaffold, or absence of a scaffold, refers to contacting a population of ASCs with a modulator of ASC aggregation as disclosed herein, includes mixing the a population of ASC with a modulator of ASC aggregation.

The terms “engineered tissue”, “engineered tissue construct”, or “tissue engineered construct” as used herein refer to a tissue or organ that is produced, in whole or in part, using tissue engineering techniques. Descriptions of these techniques can be found in, among other places, “Principles of Tissue Engineering, 2d ed.”, Lanza, Langer, and Vacanti, eds., Academic Press, 2000 (hereinafter “Lanza et al.”); “Methods of Tissue Engineering”, Atala and Lanza, eds., Academic Press, 2001 (hereinafter “Atala et al.”); Animal Cell Culture, Masters, ed., Oxford University Press, 2000, (hereinafter “Masters”), particularly Chapter 6; and U.S. Pat. No. 4,963,489 (which is incorporated herein in its entirety by reference), and related U.S. patents. By way of an example only, a “tissue engineered” myocardium refers to the artificial creation of myocardial tissue from cells, such as cardiomyocytes or cardiac progenitors, or from cells such as iPS cells which have been differentiated to become cardiomyocytes. In some embodiments, engineered tissue can comprises three-dimensional matrices and/or an appropriate scaffold such as biopolymer scaffolds as disclosed herein.

As used herein, the term “stem cells” is used in a broad sense and includes traditional stem cells, progenitor cells, pre-progenitor cells, reserve cells, and the like. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735 (which are incorporated herein in its entirety by reference). Descriptions of stromal cells, including methods for isolating them, may be found in, among other places, Prockop, Science, 276:7174, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489 (which is incorporated herein in its entirety by reference).

As used herein, the terms “iPS cell” and “induced pluripotent stem cell” are used interchangeably and refers to a pluripotent cell artificially derived (e.g., induced by complete or partial reversal) from an undifferentiated cell (e.g. a non-pluripotent cell) or a somatic cell such as a differentiated somatic cell. iPS cells are capable of self-renewal and differentiation into cell fate-committed stem cells, including neural stem cells, as well as various types of mature cells.

The term “derived from” used in the context of a cell derived from another cell means that a cell has stemmed (e.g. changed from or produced by) a cell which is a different cell type. In some instances, for e.g. a cell derived from an iPS cell refers to a cell which has differentiated from an iPS cell. Alternatively, a cell can be converted from one cell type to a different cell type by a process referred to as transdifferention or direct reprogramming. Alternatively, in the terms of iPS cells, a cell (e.g. iPS cell) can be derived from a differentiated cell by a process referred to in the art as dedifferentiation or reprogramming.

The term “Relevant Cells”, as used herein refers to cells that are appropriate for being combined with an admixture comprising ASCs and a modulator of ASC aggregation as disclosed herein depends on the use of the admixture comprising ASCs and a modulator of ASC aggregation for transplantation into a subject. For example, Relevant Cells that are appropriate for the repair, restructuring, or repopulation of damaged liver may include, without limitation, hepatocytes, biliary epithelial cells, Kupffer cells, fibroblasts, and the like. Exemplary Relevant Cells for incorporation into prevascularized constructs include neurons, myocardiocytes, myocytes, chondrocytes, pancreatic acinar cells, islets of Langerhans, osteocytes, hepatocytes, Kupffer cells, fibroblasts, myocytes, myoblasts, satellite cells, endothelial cells, adipocytes, preadipocytes, biliary epithelial cells, and the like. These types of cells may be isolated and cultured by conventional techniques known in the art. Exemplary techniques can be found in, among other places, Atala et al., particularly Chapters 9 32; Freshney, Culture of Animal Cells A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022 (which are incorporated herein in its entirety by reference).

The term “isolated” when used in reference to cells, refers to a single cell of interest, or a heterogeneous population of cells of interest such as ASCs, at least partially isolated from other cell types or other cellular material with which it naturally occurs in the tissue of origin (e.g., adipose tissue). Stated another way, isolated ASCs are substantially free of adipocytes and red blood cells and clonal populations of connective tissue stem cells, and are substantially devoid of cells such as extracellular matrix material and cells from adipose tissue. A sample of ASCs which is “substantially pure” when it is at least 60%, or at least 75%, or at least 90%, and, in certain cases, at least 99% free of cells of adipose tissue other than cells of interest. For clarity, the cells of interest in a heterogeneous population of cells of a ASC population include, for example but are not limited to endothelial cells, mesenchymal stem cells, fibroblasts, smooth muscle cells, pericytes and adipose-derived stem cells. Purity can be measured by any appropriate method, for example, by fluorescence-activated cell sorting (FACS), or other assays which distinguish cell types. The term “enriching” is used synonymously with “isolating” cells, and means that the yield (fraction) of cells of one type is increased by at least 10% over the fraction of cells of that type in the starting culture or preparation.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of ASCs, refers to a population of cells that contain fewer than about 20%, more preferably fewer than about 15%, 10%, 8%, 7%, most preferably fewer than about 5%, 4%, 3%, 2%, 1%, or less than 1%, of cells that are not ASCs as defined by the terms herein.

As used herein, the term “purified”, relates to an enrichment of a cell, cell type, molecule, or compound relative to other components normally associated with the cell, cell type, molecule, or compound in a native environment. The term “purified” does not necessarily indicate that complete purity of the particular cell, cell type, molecule, or compound has been achieved during the process. A “highly purified” population of ASCs as used herein refers to a population of ASCs that is greater than 90% pure (i.e. the highly purified population of ASCs comprises at least 90% cells of SVF population (i.e. endothelial cells, mesenchymal stem cells, fibroblasts, smooth muscle cells, pericytes and adipose-derived stem cells) relative to non-ASCs such as red blood cells, adipocytes and cells of the extracellular matrix of adipose tissue).

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of ASCs as used herein refers to a population of cells that has been removed and separated from a non-ASCs in a mixed or heterogeneous population of ASCs and non-ASCs. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

The terms “subject” and “individual” are used interchangeably herein, and refer to an animal, for example a human, to whom a ASCs can be harvested from, or a subject whom an admixture of ASCs and a modulator of ASC aggregation can be administered or transplanted into for treatment, including prophylactic treatment, using the methods and compositions described herein. For treatment of conditions or disease states which are specific for a specific animal such as a human subject, the term “subject” refers to that specific animal. In some embodiments, the subject is a human subject. The terms “non-human animals” and “non-human mammals” are used interchangeably herein, and include mammals such as rats, mice, rabbits, sheep, cats, dogs, cows, pigs, and non-human primates. In some embodiments, the invention encompasses recipient subjects which are a different mammalian subject to the donor subject. As an illustrative example only, a donor subject may be a pig subject, and the recipient subject can be a human subject.

The term “mammal” or “mammalian” are used interchangeably herein, are intended to encompass their normal meaning. While the invention is most desirably intended for efficacy in humans, it may also be employed in domestic mammals such as canines, felines, and equines, as well as in mammals of particular interest, e.g., zoo animals, farmstock, transgenic animals, rodents and the like.

As used herein, the term “donor” refers to a subject from which a organ, tissue or cell to be transplanted is harvested from.

As used herein, the term “recipient” refers to a subject which will receive a transplanted organ, tissue or cell.

The term “three-dimensional matrix” is used in the broad sense herein and refers to a composition comprising a biocompatible matrix, scaffold, or the like. The three-dimensional matrix may be liquid, gel, semi-solid, or solid at 25° C. The three-dimensional matrix may be biodegradable or non-biodegradable. In some embodiments, the three-dimensional matrix is biocompatible, or bioresorbable or bioreplacable. Exemplary three-dimensional matrices include polymers and hydrogels comprising collagen, fibrin, chitosan, MATRIGEL™, polyethylene glycol, dextrans including chemically crosslinkable or photocrosslinkable dextrans, processed tissue matrix such as submucosal tissue and the like. In certain embodiments, the three-dimensional matrix comprises allogeneic components, autologous components, or both allogeneic components and autologous components. In certain embodiments, the three-dimensional matrix comprises synthetic or semi-synthetic materials. In certain embodiments, the three-dimensional matrix comprises a framework or support, such as a fibrin-derived scaffold.

The term “biodegradable” as used herein denotes a composition that is not biologically harmful and can be chemically degraded or decomposed by natural effectors (e.g., weather, soil bacteria, plants, animals).

The term “bioresorbable” refers to the ability of a material to be reabsorbed over time in the body (e.g. in vivo) so that its original presence is no longer detected once it has been reabsorbed.

The term “bioreplaceable” as used herein, and when used in the context of an implant, refers to a process where de novo growth of the endogenous tissue replaces the implant material. A bioreplacable material as disclosed herein does not provoke an immune or inflammatory response from the subject and does not induce fibrosis. A bioreplaceable material is distinguished from bioresorbable material in that bioresorbable material is not replaced by de novo growth by endogenous tissue.

The terms “processed tissue matrix” and “processed tissue material” are used interchangeably herein, to refer to native, normally cellular tissue that as been procured from an animal source, for example a mammal, and mechanically cleaned of attendant tissues and chemically cleaned of cells and cellular debris, and rendered substantially free of non-collagenous extracellular matrix components. In some embodiments, the processed tissue matrix can further comprise non-cellular material naturally secreted by cells, such as intestinal submucosa cells, isolated in their native configuration with or without naturally associated cells.

As used herein the term “submucosal tissue” refers to natural extracellular matrices, known to be effective for tissue remodelling, that have been isolated in their native configuration. The submucosal tissue can be from any animal, for example a mammal, such as but not limited to, bovine or porcine submucosal tissue. In some embodiments, the submucosal tissue is derived from a human, such as the subject into which it is subsequently implanted (e.g. autograft transplantation) or from a different human donor (e.g. allograft transplantation). The submucosa tissue can be derived from intestinal tissue (autograft, allograft, and xenograft), stomach tissue (autograft, allograft, and xenograft), bladder tissue (autograft, allograft, and xenograft), alimentary tissue (autograft, allograft, and xenograft), respiratory tissue (autograft, allograft, and xenograft) and genital tissue (autograft, allograft, and xenograft), and derivatives of liver tissue (autograft, allograft, and xenograft), including for example liver basement membrane and also including, but not limited to, dermal extracellular matrices (autograft, allograft, and xenograft) from skin tissue.

The term “scaffold” is also used in a broad sense herein. Thus scaffolds include a wide variety of three-dimensional frameworks, for example, but not limited to a mesh, grid, sponge, foam, or the like.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least about 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” or “activate” are all used herein to generally mean an increase by a statistically significant amount; for the avoidance of any doubt, the terms “increased”, “increase” or “enhance” or “activate” means an increase of at least about 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “substantially” as used herein means a proportion of at least about 60%, or preferably at least about 70% or at least about 80%, or at least about 90%, at least about 95%, at least about 97% or at least about 99% or more, or any integer between 70% and 100%.

The term “gene” as used herein refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript, which are termed “5′ untranslated regions” or 5′UTR and 3′ untranslated regions (3′UTR) respectively. These sequences are also referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation. Expression of a gene, for example of a genetically engineered cell (e.g. a genetically engineered ASC) can be achieved by introducing a gene which is operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., 3′UTR, 5″UTR, introns) between individual coding regions (i.e., exons). The term “structural gene” as used herein is intended to mean a DNA sequence that is transcribed into mRNA which is then translated into a sequence of amino acids characteristic of a specific polypeptide.

The term “gene product(s)” as used herein refers to include RNA transcribed from a gene, or a polypeptide encoded by a gene or translated from RNA.

The term “expression” as used herein refers to the biosynthesis of a gene product, preferably to the transcription and/or translation of a nucleotide sequence, for example an endogenous gene or a heterologous gene, in a cell. For example, in the case of a heterologous nucleic acid sequence, expression involves transcription of the heterologous nucleic acid sequence into mRNA and, optionally, the subsequent translation of mRNA into one or more polypeptides. Expression also refers to biosynthesis of a RNAi molecule, which refers to expression and transcription of an RNAi agent such as siRNA, shRNA, and antisense DNA but does not require translation to polypeptide sequences.

The term “expression construct” and “nucleic acid construct” as used herein are synonyms and refer to a nucleic acid sequence capable of directing the expression of a particular nucleotide sequence, such as the heterologous target gene sequence in an appropriate host cell (e.g., a mammalian cell). If translation of the desired heterologous target gene is required, it also typically comprises sequences required for proper translation of the nucleotide sequence. The coding region may code for a protein of interest but may also code for a functional RNA of interest, for example antisense RNA, dsRNA, or a nontranslated RNA, in the sense or antisense direction. The nucleic acid construct as disclosed herein can be chimeric, meaning that at least one of its components is heterologous with respect to at least one of its other components.

The term “agent” refers to any entity which is normally absent or not present at the levels being administered, in the cell. Agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “a reduction” of the level of a gene, included a decrease in the level of a protein or mRNA means in the cell or organism. As used herein, “at least a partial reduction” of the level of an agent (such as a RNA, mRNA, rRNA, tRNA expressed by the target gene and/or of the protein product encoded by it) means that the level is reduced at least 25%, preferably at least 50%, relative to a cell or organism lacking the RNAi agent as disclosed herein. As used herein, “a substantial reduction” of the level of an agent such as a protein or mRNA means that the level is reduced relative to a cell or organism lacking a chimeric RNA molecule of the invention capable of reducing the agent, where the reduction of the level of the agent is at least 75%, preferably at least 85%. The reduction can be determined by methods with which the skilled worker is familiar. Thus, the reduction of the transgene protein can be determined for example by an immunological detection of the protein. Moreover, biochemical techniques such as Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) to detect transgene protein or mRNA. Depending on the type of the reduced transgene, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford MM (1976) Analyt Biochem 72:248-254).

The terms “heterologous target gene” or “heterologous gene sequence” are used interchangeably herein refers to any nucleic acid (e.g., gene sequence) which is introduced into the genome of a cell. Heterologous gene sequences may include gene sequences found in that cell so long as the introduced gene to be expressed at different levels as compared to the level naturally occurring in the host cell and/or contains some modification (e.g., a point mutation, the presence of a selectable marker gene, etc.) relative to the naturally-occurring gene, or is not expressed at the same level normally in the cells as compared to the level which is being induced. A heterologous target gene can be present in the cell but not at the levels being expressed, or the nucleic acid sequence has been modified by experimental manipulations, such as example, modification being a substitution, addition, deletion, inversion or insertion of one or more nucleotide residues, for example modified by non-natural, synthetic or “artificial” methods such as, for example mutagenesis or the nucleic acid is not located in its natural or native genetic environment. Such methods for nucleic acid modification have been described (U.S. Pat. No. 5,565,350; WO 00/15815 which is incorporated herein by reference). In some instances a heterologous target gene includes, but are not limited to, coding sequences of heterologous genes or structural genes (e.g., reporter genes, selection marker genes, drug resistance genes, growth factors, etc.), and non-coding regulatory sequences which do not encode an mRNA or protein product, (e.g., promoter sequence, polyadenylation sequence, termination sequence, enhancer sequence, RNAi molecules etc.). A nucleic acid sequence of interest may preferably encode for a heterologous gene, for example a valuable trait, for example but not limited to, a heterologous gene encoding a toxin protein, or fragment thereof for use in cancer therapeutics etc. In some instances, a heterologous target gene is not endogenous to or not naturally associated with the cell into which it is introduced, but has been obtained from another cell. Heterologous target genes also includes an endogenous DNA sequence, which contains some modification, non-naturally occurring multiple copies of a endogenous DNA sequence, or a DNA sequence which is not naturally associated with another DNA sequence physically linked thereto. Generally, although not necessarily, heterologous target genes encodes RNA and proteins that are not normally produced by the cell into which it is expressed.

The terms “target”, “target gene” and “target nucleotide sequence” are used equivalently herein and refers to a target gene can be any gene of interest present in an organism. A target gene may be endogenous or introduced. For example, a target gene is a gene of known function or is a gene whose function is unknown, but whose total or partial nucleotide sequence is known. Alternatively, the function of a target gene and its nucleotide sequence are both unknown. A target gene can be a native gene of the eukaryotic cell or can be a heterologous gene which has previously been introduced into the eukaryotic cell or a parent cell of said eukaryotic cell, for example by genetic transformation. A heterologous target gene can be stably integrated in the genome of the eukaryotic cell or is present in the eukaryotic cell as an extrachromosomal molecule, e.g. as an autonomously replicating extrachromosomal molecule. A target gene can include polynucleotides comprising a region that encodes a polypeptide or polynucleotide region that regulates replication, transcription, translation, or other process important in expression of the target protein; or a polynucleotide comprising a region that encodes the target polypeptide and a region that regulates expression of the target polypeptide; or non-coding regions such as the 5′ or 3′ UTR or introns. A target gene may refer to, for example, an mRNA molecule produced by transcription a gene of interest.

The term “endogenous” nucleotide sequence refers to a nucleotide sequence, which is present in the genome of the cell under normal conditions, i.e. a nucleotide sequence which present normally in the cell and is not introduced into the cell or by other genetic manipulation strategies. A nucleic acid sequence referred to as a “non-endogenous” or “synthetic” sequence refers to a sequence, where the entire sequence is not found in the cell to which the nucleic acid is introduced. In some embodiments, the RNAi target site is a non-endogenous or synthetic sequence, meaning the entire sequence is not found within the cell that the nucleic acid construct is introduced into.

As used herein, “gene silencing” or “gene silenced” in reference to an activity of a RNAi molecule, for example a siRNA or miRNA refers to a decrease in the mRNA level in a cell for a heterologous target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%. As used herein, the “reduced” or “gene silencing” refers to lower, preferably significantly lower, more preferably the expression of the nucleotide sequence is not detectable.

The term “double-stranded RNA” molecule, “RNAi molecule”, or “dsRNA” molecule as used herein refers to a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. In some embodiments, the terms refer to a double-stranded RNA molecule capable, when expressed, is at least partially reducing the level of the mRNA of the heterologous target gene. In particular, the RNAi molecule is complementary to a synthetic RNAi target sequence located in a non-coding region of the heterologous target gene. As used herein, “RNA interference”, “RNAi”, and “dsRNAi” are used interchangeably herein refer to nucleic acid molecules capable of gene silencing.

As used herein, the term “RNAi” refers to any type of interfering RNA, including but are not limited to, siRNAi, shRNAi, stRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “siRNA” also refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 10-50 nucleotides in length (e.g., each complementary sequence of the double stranded siRNA is about 10-22 nucleotides in length, and the double stranded siRNA is about 10-22 base pairs in length, preferably about 19-22 base nucleotides, preferably about 17-19 nucleotides in length, e.g., 10, 11, 12, 13, 14, 15, 16, 17, 18, 19, 20, 21, or 22 nucleotides in length).

As used herein “shRNA” or “small hairpin RNA” (also called stem loop) is a type of siRNA. In one embodiment, these shRNAs are composed of a short, e.g. about 10 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches. In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base pairing may not include any mismatches.

As used herein the term “hairpin RNA” refers to any self-annealing double stranded RNA molecule. In its simplest representation, a hairpin RNA consists of a double stranded stem made up by the annealing RNA strands, connected by a single stranded RNA loop, and is also referred to as a “pan-handle RNA”. However, the term “hairpin RNA” is also intended to encompass more complicated secondary RNA structures comprising self-annealing double stranded RNA sequences, but also internal bulges and loops. The specific secondary structure adapted will be determined by the free energy of the RNA molecule, and can be predicted for different situations using appropriate software such as FOLDRNA (Zuker and Stiegler (1981) Nucleic Acids Res 9(1):133-48; Zuker, M. (1989) Methods Enzymol. 180, 262-288).

As used herein, the term “amino acid sequence” refers to a list of abbreviations, letters, characters or words representing amino acid residues Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “nucleic acids” and “nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA”, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. shRNAs also can comprise non-natural elements such as non-natural bases, e.g., ionosin and xanthine, normatural sugars, e.g., 2′-methoxy ribose, or non-natural phosphodiester linkages, e.g., methylphosphonates, phosphorothioates and peptides. The term “nucleic acid” or “oligonucleotide” or “polynucleotide” are used interchangeably herein and refers to at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions.

The term “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O— and N— alkylated nucleotides, e.g. N6-methyl adenosine are suitable. The 2′ OH— group can be replaced by a group selected from H. OR, R. halo, SH, SR, NH₂, NHR, NR₂ or CN, wherein R is C-C6 alkyl, alkenyl or alkynyl and halo is F, Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

The terms “polypeptide”, “peptide”, “oligopeptide”, “polypeptide”, “gene product”, “expression product” and “protein” are used interchangeably herein to refer to a polymer or oligomer of consecutive amino acid residues.

The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence. The term transgenic when referring to a cell, tissue or organisms means transformed, preferably stably transformed, with a recombinant DNA molecule that preferably comprises a suitable promoter operatively linked to a DNA sequence of interest.

The term “vector” is used interchangeably with “plasmid” to refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression encoding the DNA. A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affection.

The term “pathology” as used herein, refers to symptoms, for example, structural and functional changes in a cell, tissue or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

As used herein, the terms “treat” or “treatment” or “treating” refers to both therapeutic treatment and prophylactic or preventative measures, wherein the object is to prevent or slow the development of the disease. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “effective amount” as used herein refers to the amount of therapeutic agent of pharmaceutical composition (e.g. a transplant mixed with ASCs, or transplant encapsulated with ASCs) to reduce at least one or more symptom(s) of the disease or disorder, and relates to a sufficient amount of pharmacological composition to provide the desired effect. The phrase “therapeutically effective amount” as used herein, (e.g., amount of a transplant mixed with ASCs, or transplant encapsulated with ASCs) means a sufficient amount of the composition to treat a disorder, at a reasonable benefit/risk ratio applicable to any medical treatment. The term “therapeutically effective amount” therefore refers to an amount of the composition as disclosed herein (e.g. transplant mixed with ASCs, or transplant encapsulated with ASCs) that is sufficient to effect a therapeutically or prophylatically significant reduction in a symptom or clinical marker associated with a dysfunction or disorder when administered to a typical subject who has a condition, disease or disorder to be treated.

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

As used herein, the terms “administering,” “introducing” and “transplanting” are used interchangeably and refer to the placement of an admixture comprising a population of ASCs and a modulator of ASC aggregation, e.g. ASCs plus heparin as described herein into a subject by a method or route which results in at least partial localization of the ASCs at a desired site. The an admixture comprising a population of ASCs and a modulator of ASC aggregation, e.g. ASCs plus heparin can be administered by any appropriate route which results in effective treatment in the subject, e.g. administration results in delivery to a desired location in the subject where at least a portion of the transplanted ASCs remain viable. The period of viability of the cells after administration to a subject can be as short as a few hours, e.g. twenty-four hours, to a few days, to as long as several years.

The phrases “parenteral administration” and “administered parenterally” as used herein mean modes of administration of an admixture comprising a population of ASCs and a modulator of ASC aggregation, e.g. ASCs plus heparin other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of the compositions as disclosed herein, e.g. an admixture comprising a population of ASCs and a modulator of ASC aggregation, to the subject such that it enters the animal's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof.

In this specification and the appended claims, the singular forms “a,” “an,” and “the” include plural references unless the context clearly dictates otherwise, and therefore “a” and “an” are used herein to refer to one or to more than one (i.e., at least one) of the grammatical object of the article. By way of example, “an element” means one element or more than one element, and reference to a composition for delivering “an agent” includes reference to one or more agents.

Compositions or methods “comprising” one or more recited elements may include other elements not specifically recited. For example, a composition that comprises ASCs encompasses both the isolated ASCs but may also include other cell types or protein or other components. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.”

Other than in the operating examples, or where otherwise indicated, all numbers expressing quantities of ingredients or reaction conditions used herein should be understood as modified in all instances by the term “about.” The term “about” is used herein to mean approximately, roughly, around, or in the region of. When the term “about” is used in conjunction with a numerical range, it modifies that range by extending the boundaries above and below the numerical values set forth. The term “about” when used in connection with percentages will mean±1%.

### I. Adipose-Derived Stromal Cells (ASCs)

One aspect of the present invention relates to an admixture comprising a population of ASCs in combination with a modulator of ASC aggregation. ASCs are also commonly referred to in the art as s “ADSCs” or stromal vascular fraction cells (SVFs) and refer to a heterologous population of cells derived from digestion of adipose tissue. This population of cells is referred to as ASCs or “adipose derived stromal cells” herein.

Without wishing to be bound to theory, adipose tissue plays an important and overlooked role in the normal development and physiology of humans and other mammalian species. Many different kinds of fat exist. The most common type is white adipose tissue, located under the skin (subcutaneous fat), within the abdominal cavity (visceral fat) and around the reproductive organs (gonadal fat). Less common in the adult human is brown adipose tissue, which plays an important role in generating heat during the neonatal period; this type of fat is located between the shoulder blades (interscapular), around the major vessels and heart (periaortic and pericardial), and above the kidney (suprarenal). Adipose tissue also encompasses yellow fat. Adipose tissue is found throughout the body of an animal, including humans, and is present in the omementurm, bone marrow, subcutaneous space and surrounding most organs.

Adult ASC or human adipose tissue-derived adult stromal cells represent a cell source that can be harvested routinely with minimal risk or discomfort to the subject. They can be expanded ex vivo, differentiated along unique lineage pathways, genetically engineered, and re-introduced into individuals as either autologous or allogenic transplantation.

A population of ASCs as described herein, comprise is a heterologous population of cells comprising at least one or at least 2 or the following population of cells; endothelial cells, mesenchymal stem cells, fibroblasts, smooth muscle cells, pericytes and adipose-derived stem cells, as well as additional other cell types not listed. In some embodiments, adipose-derived stromal cells refers to a substantially pure population of adipose-derived stem cells. ASC useful in the methods of the present invention have the ability to differentiate into various cell types, including, but no limited to, adipocytes, chondrocytes, and osteoblasts, as well as provide fully differentiated and functional cells for research, transplantation, and development of tissue engineering products for the treatment of diseases and disorders and traumatic injury repair.

ASCs as described herein can be isolated from adipose tissue using methods previously described (Zuk et al., Tissue Engineering 7:211, 2001; Katz et al., Stem Cells 23:412, 2005). However, one of ordinary skill in the art will appreciate that culture conditions such as cell seeding ASCs densities can be selected for each experimental condition or intended use.

ASCs can be cultured according to method commonly known in the art to induce the ASCs to give rise to cells having a mesodermal, ectodermal or endodermal lineage. After culturing ASCs in the differentiating-inducing medium for a suitable time (e.g., several days to a week or more), the ASCs can be assayed to determine whether, in fact, they have acquired the desired lineage.

Methods to characterize a population of ASCs, include, but are not limited to, histological, morphological, biochemical and immunohistochemical methods, or using cell surface markers, or genetically or molecularly, or by identifying factors secreted by the differentiated cell, and by the inductive qualities of the differentiated ASCs. US 2002/0076400 and WO 00/53795 (which are incorporated herein by reference) describe the production of multipotent cell populations from human adipose tissue. Said cell populations can be differentiated into adipocytes, osteoblasts, chondrocytes, and myocytes. The publications indicate that some of the cells they can be maintained in culture in vitro for at least 15 cell transfers without losing their multipotent character. U.S. Pat. No. 6,800,480, which is incorporated herein by reference, describes methods and materials for growing primate-derived primordial stem cells in a feeder cell-free culture system.

For example, molecular markers that characterize mesodermal cell that differentiate from the ASCs of the invention, include, but are not limited to, MyoD, myosin, alpha-actin, brachyury, FOG, tbx5 FoxF1, Nkx-2.5. Mammalian homologs of the above mentioned markers are preferred.

Molecular markers that characterize ectodermal cell that differentiate from the ASCs of the invention, include for example, but are not limited to N-CAM, GABA and epidermis specific keratin. Mammalian homologs of the above mentioned markers are preferred. Molecular markers that characterize endodermal cells that differentiate from the ADSCs include for example, but are not limited to, Xhbox8, Endol, Xhex, Xcad2, Edd, EF1-alpha, HNF3-beta, LFABP, albumin, insulin. Mammalian homologs of the above mentioned markers are preferred.

Other techniques useful for isolating and characterizing the ASCs as described herein include fractionating cells using cell markers. The immunophenotype of the ASCs based on flow cytometry include Stromal cell-associated markers, such as CD13, CD29, CD34, CD44, CD63, CD73, CD90, CD166, as well as aldehyde dehydrogenase and the multidrug-resistance transport protein (ABCG2). ASCs can also express endothelial cell-associated markers, such as for example but not limited to, CD31, CD144 or VE-cadherin, vascular endothelial growth factor receptor 2, von Willebrand factor.

ASCs also express a number of adhesion and surface proteins. These include cell surface markers such as CD9; CD29 (integrin beta 1); CD44 (hyaluronate receptor); CD49d,e (integrin alpha 4, 5); CD54 (ICAM1); CD55 (decay accelerating factor); CD105 (endoglin); CD106 (VCAM-1); CD166 (ALCAM) and HLA-ABC (Class I histocompatibility antigen); and cytokines such as interleukins 6, 7, 8, 11; macrophage-colony stimulating factor; GM-colony stimulating factor; granulocyte-colony stimulating factor; leukemia inhibitory factor; stem cell factor and bone morphogenetic protein. Many of these proteins have the potential to serve a hematopoietic supportive function and all of them are shared in common by bone marrow stromal cells.

In some embodiments, the ASCs used in the methods, compositions and admixtures as disclosed herein are genetically engineered. In certain embodiments, an ASC is genetically engineered to express at least one cytokine, chemokine, antibiotic, drug, analgesic, anti-inflammatory, or immune suppressants, or the like. Exemplary cytokines include angiogenin, vascular endothelial growth factor (VEGF, including, but not limited to VEGF-165), interleukins, fibroblast growth factors, for example, but not limited to, FGF-1 and FGF-2, hepatocyte growth factor, (HGF), transforming growth factor beta (TGF-.beta.), endothelins (such as ET-1, ET-2, and ET-3), insulin-like growth factor (IGF-1), angiopoietins (such as Ang-1, Ang-2, Ang-3/4), angiopoietin-like proteins (such as ANGPTL1, ANGPTL-2, ANGPTL-3, and ANGPTL-4), platelet-derived growth factor (PDGF), including, but not limited to PDGF-AA, PDGF-BB and PDGF-AB, epidermal growth factor (EGF), endothelial cell growth factor (ECGF), including ECGS, platelet-derived endothelial cell growth factor (PD-ECGF), placenta growth factor (PLGF), and the like. The skilled artisan will understand that the choice of chemokines and cytokine fragments to be expressed by an engineered ASC will depend, in part, on the use of the admixture comprising a ASC population and modulator of ASC aggregation (e.g. heparin) for transplantation into a subject.

In certain embodiments, a composition or admixture comprising a population of ASCs and a modulator of ASC aggregation, comprises the ASCs on a 3D biocompatible matrix. In some embodiments, a population of ASCs on a 3D biocompatible matrix comprise at least one genetically engineered ASC. In certain embodiments, a genetically engineered ASC cell will constitutively express or inducibly express at least one gene product encoded by the at least one genetically engineered cell due to the genetic alterations within the at least one genetically engineered cell induced by techniques known in the art. Descriptions of exemplary genetic engineering techniques can be found in, among other places, Ausubel et al., Current Protocols in Molecular Biology (including supplements through March 2002), John Wiley & Sons, New York, N.Y., 1989; Sambrook et al., Molecular Cloning: A Laboratory Manual, 2nd Ed., Cold Spring Harbor Laboratory Press, Cold Spring Harbor, N.Y., 1989; Sambrook and Russell, Molecular Cloning: A Laboratory Manual, 3rd Ed., Cold Spring Harbor Laboratory Press, Cold Spring Harbor, N.Y., 2001; Beaucage et al., Current Protocols in Nucleic Acid Chemistry, John Wiley & Sons, New York, N.Y., 2000 (including supplements through March 2002); Short Protocols in Molecular Biology, 4th Ed., Ausbel, Brent, and Moore, eds., John Wiley & Sons, New York, N.Y., 1999; Davis et al., Basic Methods in Molecular Biology, McGraw Hill Professional Publishing, 1995; Molecular Biology Protocols (see the highveld.com website), and Protocol Online (protocol-online.net). Exemplary gene products for genetically modifying the genetically engineered SVF cells of the invention include, plasminogen activator, soluble CD4, Factor VIII, Factor IX, von Willebrand Factor, urokinase, hirudin, interferons, including α-, β- and γ-interferon, tumor necrosis factor, interleukins, hematopoietic growth factor, antibodies, glucocerebrosidase, adenosine deaminase, phenylalanine hydroxylase, human growth hormone, insulin, erythropoietin, VEGF, angiopoietin, hepatocyte growth factor, PLGF, and the like.

### II. Methods to Obtain ASCs.

Adipose tissue is readily accessible and abundant in many individuals. Obesity is a condition of epidemic proportions in the United States, where over 50% of adults exceed the recommended BMI. Adult adipose-derived stromal cells (ASC) can be harvested from a subject, for example, using the methods and devices as disclosed in U.S. Pat. No. 7,270,996, which is incorporated herein by reference. Additionally, adult adipose-derived stromal cells (ASC) can be obtained and cultured according to the culture conditions as disclosed, for example, in U.S. Patent Application 2008/0248003 which is incorporated herein by reference.

ASCs useful in the methods of invention can be isolated and identified by a variety of methods known to those skilled in the art such as described in US Patent Application 2003/0082152, International application WO00/53795 and U.S. Pat. Nos. 4,820,626, 4,883,755, 5,035,708 and 5,957,972 and 7,470,537, which are all incorporated herein in their entirety by reference. Alternatively, the process of isolating the ASC enriched fraction can be performed using a suitable device, many of which are known in the art (see, e.g., U.S. Pat. No. 5,786,207 which is incorporated herein in its entirety by reference). Such devices can mechanically achieve the washing and dissociation steps if obtaining ASCs from adipose tissue.

In some embodiments, adipose tissue is isolated from a mammalian subject, preferably a human subject. In some embodiments, a source of adipose is subcutaneous adipose tissue. In some embodiments, a source of adipose tissue is omental adipose tissue. In humans, the adipose tissue is typically isolated by liposuction. In some embodiments, where engineered ASC are to be transplanted into a human subject, it is preferable that the adipose tissue be isolated from that same subject to provide for an autologous transplant. Alternatively, the transplanted ASCs are allogeneic.

One can use any method for the isolation, expansion to obtain ASCs for the methods, compositions and admixtures as disclosed herein. For example, one can use any procedure as previously reported, for example, methods as disclosed in Burris et al. 1999, Mol Endocrinol 13:410-7; Erickson et al. 2002, Biochem Biophys Res Commun. Jan. 18, 2002; 290(2):763-9; Gronthos et al. 2001, Journal of Cellular Physiology, 189:54-63; Halvorsen et al. 2001, Metabolism 50:407-413; Halvorsen et al. 2001, Tissue Eng. 7(6):729-41; Harp et al. 2001, Biochem Biophys Res Commun 281:907-912; Saladin et al. 1999, Cell Growth & Diff 10:43-48; Sen et al. 2001, Journal of Cellular Biochemistry 81:312-319; Zhou et al. 1999, Biotechnol. Techniques 13: 513-517. Adipose tissue-derived stromal cells are obtained from minced human adipose tissue by collagenase digestion and differential centrifugation [Halvorsen et al. 2001, Metabolism 50:407-413; Hauner et al. 1989, J Clin Invest 84:1663-1670; Rodbell et al. 1966, J Biol Chem 241:130-139].

It is well documented that adipocytes are a replenishable cell population. Even after surgical removal by liposuction or other procedures, it is common to see a recurrence of adipocytes in an individual over time. This suggests that adipose tissue contains stromal stem cells and/or precursors that are capable of self-renewal.

However obtained, the adipose tissue is processed to separate the ASCs of the invention from the remainder of the adipose tissue. The ASC population that contains a heterogeneous population of mesenchymal stem cells, fibroblasts, smooth muscle cells and pericytes and adipose-derived stem cells is obtained by washing the obtained adipose tissue with a physiologically-compatible solution, such as phosphate buffered saline (PBS). The washing step typically consists of rinsing the adipose tissue with PBS, agitating the tissue, and allowing the tissue to settle. In addition to washing, the adipose tissue is dissociated. The dissociation can occur by enzyme degradation and neutralization. Alternatively, or in conjunction with such enzymatic treatment, other dissociation methods can be used such as mechanical agitation, sonic energy, or thermal energy. Three layers form after the washing, dissociation, and settling steps. The top layer is a free lipid layer. The middle layer includes the lattice and adipocyte aggregates. The middle layer is referred to as an “adipose-derived lattice enriched fraction.”

The bottom layer contains the ASC population. The bottom layer is further processed to isolate the ASCs as disclosed herein. The cellular fraction of the bottom layer is concentrated into a pellet. One method to concentrate the cells includes centrifugation.

The bottom layer is centrifuged and the pellet is retained. The pellet is designated the adipose-derived stromal cell population which includes the adipose-derived stem cells as well as other cells in the ASC population. The ASC population can also contain erythrocytes (RBCs). In a preferred method the RBCs are lysed and removed. Methods for lysis and removal of RBCs are well known in the art (e.g., incubation in hypotonic medium). However, the RBCs are not required to be removed from the ADSC-EF.

The pellet is resuspended and can be washed (in PBS), centrifuged, and resuspended one or more successive times to achieve greater purity of the ASCs. The ASC population as disclosed herein is a heterogenous population of cells which include, among other cells, adipose-derived stem cells (ADSCs). The cells of the washed and resuspended pellet are ready for genetic manipulation and/or subsequent transplantation into a subject.

The ASCs in the resuspended pellet can be separated from other cells of the resuspended pellet by methods that include, but are not limited to, cell sorting, size fractionation, granularity, density, molecularly, morphologically, and immunohistologically. The immunophenotype of the adipose-derived stromal cells based on flow cytometry include Stromal cell-associated markers, such as CD13, CD29, CD34, CD44, CD63, CD73, CD90, CD166, as well as aldehyde dehydrogenase and the multidrug-resistance transport protein (ABCG2). ASC can also express endothelial cell-associated markers, such as for example but not limited to, CD31, CD144 or VE-cadherin, vascular endothelial growth factor receptor 2, von Willebrand factor.

In one embodiment, the ASCs are separated from the other cells on the basis of cell size and granularity where ASCs are small and agranular. Alternatively, a molecular method for separating the ASCs from the other cells of the pellet is by assaying the length of the telomere. Adipose-derived stem cells (ADSCs) tend to have longer telomeres than differentiated cells.

In another embodiment, a biochemical method for separating the ASCs from the other cells of the pellet is used by assaying telomerase activity. Telomerase activity can serve as a stem cell-specific marker.

In still another embodiment, the ASCs are separated from the other cells of the pellet immunohistochemically, for example, by panning, using magnetic beads, or affinity chromatography.

Alternatively, the process of isolating the ADSC enriched fraction with the ASCs is with a suitable device, many of which are known in the art (see, e.g., U.S. Pat. No. 5,786,207 (which is incorporated herein in its entirety by reference)). Such devices can mechanically achieve the washing and dissociation steps. In some embodiments, where ASCs are isolated using a device, such as for example, as disclosed in 5,786,207, a modulator of ASC aggregation (such as heparin or other) can be directly added to the isolated population of ASCs at one or more of any of the following timepoints: (i) a modulator of ASC aggregation (such as heparin or other) can be added to the ASCs using the device, where at least one the modulator of ASC is in resuspension media bag which is used to rinse and resuspend the ASC cells. (ii) a modulator of ASC aggregation (such as heparin or other) can be added to the ASCs using the device, where at least one the modulator of ASC is added to the ASC cells, where the modulator of ASCs is present in a separate container (e.g. as a separate “bag”) to the resuspension media bag or wash bag, and is inserted into the hardware device, where the device is configured to added modulators of ASC to the ASCs or media at customized rinse/wash times during the processing of cells. In such an embodiment, modulators of ASC aggregation can be added at various and multiple timepoints in the preparation and isolation of a population of ASC. (iii) in an alternative embodiment, a modulator of ASC aggregation (such as heparin or other) is stored in a separate container (e.g. as a separate “bag”) in the device, and the modulators of ASCs can be added to the ASC population using the device, where the device is configured to add at least one the modulator of ASC to the final resuspensate of ASC during lobe washing or resuspension steps, where the modulator of ASCs can be combined with default media or used as a separate media just for resuspension. In all embodiments, a device which is used to harvest and isolate a population of ASC can be configured (e.g. by modifications to hardware and/or software) to add at least one modulator of ASC to a population of ASCs, either at one or more points during the isolation and washing procedure, and/or at the final resuspension of the ASC population in a resuspension buffer. In some embodiments, a device which is used to harvest and isolate a population of ASC can be configured (e.g. by modifications to hardware and/or software) to titrate or modify the amount of at least one modulator of ASC which is added to a population of ASCs, depending on, for example, the type of modulator of ASC aggregation (e.g. heparin versus other types of modulators of ASC aggregation), the concentration of modulator of ASC aggregation and the timepoint in the isolation/washing procedure in which the modulator of ASC aggregation is being added. In some embodiments, the wash buffers for washing the ASCs comprises one or more modulators of ASC aggregation. In some embodiments, a resuspension buffer for resuspending ASC comprises one or modulators of ASC aggregation.

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there about at least 8.6×10⁴ or at least 8.6×10⁶ stromal cells per gram of adipose tissue (Sen et al., 2001, J. Cell. Biochem., 81:312-319). Ex vivo expansion over 2 to 4 weeks yields up to 500 million stromal cells from 0.5 kilograms of adipose tissue.

Accordingly, in some embodiments engineered ASC as disclosed herein can be used immediately for transplantation or administration to a subject with cancer or cryopreserved for future autologous or allogenic applications to subjects in need thereof.

Cells described herein can be isolated from adipose tissue using methods previously described (Zuk et al., Tissue Engineering 7:211, 2001; Katz et al., Stem Cells 23:412, 2005). However, one of ordinary skill in the art will appreciate that culture conditions such as cell seeding densities can be selected for each experimental condition or intended use. Other techniques useful for isolating and characterizing the cells described herein include fractionating cells using cell markers.

US 2002/0076400 and WO 00/53795 (which are incorporated herein by reference) describe the production of multipotent cell populations from human adipose tissue. Said cell populations can be differentiated into adipocytes, osteoblasts, chondrocytes, and myocytes. The publications indicate that some of the cells can be maintained in culture in vitro for at least 15 cell passages without losing their multipotent character. U.S. Pat. No. 6,800,480, which is incorporated herein by reference, describes methods and materials for growing primate-derived primordial stem cells in a feeder cell-free culture system.

Adipose tissue derive stromal cells useful in the methods of invention may be isolated by a variety of methods known to those skilled in the art. For example, such methods are described in U.S. Pat. No. 6,153,432 incorporated herein in its entirety. In a preferred method, adipose tissue is isolated from a mammalian subject, preferably a human subject. A preferred source of adipose tissue is omental adipose tissue. In humans, the adipose tissue is typically isolated by liposuction. If the cells of the invention are to be transplanted into a human subject, it is preferable that the adipose tissue be isolated from that same subject so as to provide for an autologous transplant. Alternatively, the administered tissue may be allogenic. In one embodiment, the allogenic cells are from an individual with similar tissue antigen, or otherwise immunologically compatible individuals.

In one method of isolating adipose tissue derived stromal cells, the adipose tissue is treated with collagenase at concentrations between 0.01 to 0.5%, preferably 0.04 to 0.2%, most preferably about 0.1%, trypsin at concentrations between 0.01 to 0.5%, preferably 0.04%, most preferably about 0.2%; and/or dispase at concentrations of 0.5 ng/ml to 10 ng/ml; and/or effective concentrations of hyaluronidase or DNase; and ethylenediaminetetra-acetic acid (EDTA) at concentrations of about 0.01 to 2.0 mM, preferably at about 0.1 to about 1.0 mM, most preferably at 0.53 mM; at temperatures between 25° to 50° C., preferably between 33° to 40° C., most preferably at 37° C., for periods of between 10 minutes to 3 hours, preferably between 30 minutes to 1 hour, most preferably 45 minutes. The cells are passed through a nylon or cheesecloth mesh filter of between 20 microns to 800 microns, more preferably between 40 to 400 microns, most preferably 70 microns. The cells are then subjected to differential centrifugation directly in media or over a Ficoll or Percoll or other particulate gradient. Cells will be centrifuged at speeds of between 100 to 3000×g, more preferably 200 to 1500×g, most preferably at 500×g for periods of between 1 minutes to 1 hour, more preferably 2 to 15 minutes, most preferably 5 minutes, at temperatures of between 4° to 50° C., preferably between 20° to 40° C., most preferably at about 25° C.

Many techniques are known to those of ordinary skill in the art for measuring adipocyte differentiation, as well as the differentiation of other mesenchymal cells and those not described herein are encompassed within the techniques of the invention.

In one embodiment, adipose tissue, or ASC derived from adipose tissue, are subjected to varied culture media conditions as described herein to support growth or differentiation under serum-free or low serum conditions. One of ordinary skill in the art will appreciate that the amount of each growth factor, hormone, compound, nutrient, vitamin, etc., used may vary according to the culture conditions, amount of additional differentiation-inducing agent used, or the number of combination of agents used when more than one agent is used.

In one embodiment, ASCs derived from adipose tissue, are subjected to varied culture media conditions as described herein to support growth or differentiation under serum-free or low serum conditions. One of ordinary skill in the art will appreciate that the amount of each growth factor, hormone, compound, nutrient, vitamin, etc., used may vary according to the culture conditions, amount of additional differentiation-inducing agent used, or the number of combination of agents used when more than one agent is used.

In some embodiments, ASCs used in the methods, compositions and admixtures as disclosed herein are cultured or freshly isolated ASCs. In some embodiments, a population of freshly isolated ASCs refers to a population of ASCs which comprises a freshly isolated ASCs that has undergone little or no incubation prior to use (e.g. use in the compositions, methods or in the preparation of an admixture as disclosed herein). The skilled artisan will appreciate that an admixture comprising a population of freshly isolated ASCs and a modulator of ASC aggregation can, but need not, be incubated. In certain embodiments, an admixture comprising a population of freshly isolated ASCs and a modulator of ASC aggregation can be “incubated” subsequent to the introduction of the admixture to a subject. If the admixture is used in conjunction with a 3D-biocompatible matrix (e.g. embedded within the matrix or on the surface of the matrix), the admixture and matrix can, for example be incubated for a sufficient amount of time to allow the 3D-biocompatible matrix construct to polymerize. In other embodiments, the combination of an admixture comprising freshly isolated ASCs and a modulator of ASC aggregation with a matrix comprises a liquid three-dimensional culture, as may be appropriate for implantation by injection (see, e.g., U.S. Pat. Nos. 5,709,854 and 6,224,893 (which is incorporated herein in its entirety by reference)). Such liquid constructs may, but need not, polymerize in situ under appropriate conditions.

Cultured ASCs are typically incubated prior to mixing with a modulator of ASC aggregation. For example, but not limited to, in a humidified incubator at 37° C. and 5% CO₂. Typically such populations of cultured ASCs are incubated for a period of one hour to thirty days, but may be incubated for shorter or longer periods, as desired. The skilled artisan will appreciate that the term “cultured” may or may not refer to the use of conventional incubation methods, such as a controlled-temperature incubator.

### III. Modulators of ASC Aggregation

In some embodiments, modulators of ASC aggregation can be combined with a population of ASCs to form an admixture and transplanted into a subject in need of treatment. In some embodiments, an admixture comprising ASCs and a modulator of ASCs is stored until a time it is needed for treatment, for example, one can cryopreserved an admixture comprising an admixture comprising ASCs and a modulator of ASCs, for example storage at +4° C. or −20° C. or −80° C., using cryopreservation methods commonly known by persons of ordinary skill in the art.

In some embodiments, a subject can be treated with a population of ASCs and concurrently treated with a modulator of ASC aggregation, such that an admixture of ASCs and a modulator of ASC aggregation is not formed prior to administration. In such embodiments, a subject can be administered a population of ASCs subsequently followed by at least one modulator of ASC aggregation, as that term is defined herein. In an alternative embodiment, administration of a modulator of ASC aggregation is prior to, or concurrent with (i.e. at the same time), or subsequent to the administration of a population of ASCs.

One can use any modulator of ASC aggregation in the compositions, methods and admixtures as disclosed herein. Examples include, but are not limited to any agent which reduces or decreases the formation of aggregates of ASCs, including reduces or decreases the formation of microaggregates of ASCs in a population of ASCs. In some embodiments, a modulator of ASC aggregation reduces or decreases the number of ASC aggregates in a population of ASCs, including any agent which reduces or decreases the number of ASC microaggregates in a population of ASCs. In some embodiments, an agent which reduces the formation of, or reduces the number of ASC aggregates or ASC microaggregates in a population of ASCs is useful as a modulator of ASC aggregation for use in the compositions, methods an admixtures as disclosed herein. Accordingly, in some embodiments, an agent which decreases the formation of, or reduces the number of ASCs which collect or group together as clumps of 4 or more individual ASCs are useful as a modulator of ASC aggregation for use in the compositions, methods an admixtures as disclosed herein.

In some embodiments, a modulator of ASC aggregation is selected from any of the group of agents selected from the following group; an ionic agent (e.g., heparin), a chelating agent (e.g., EDTA, EDTA), a proteolytic agent (e.g., trypsin or dispase and functional fragments thereof), intracellularly acting agents that block expression of surface displayed attachment proteins (e.g., integins), or any other agent that modifies attachment or self-aggregative properties of ASCs, e.g. an agent which blocks the expression of an attachment protein expressed on the cell surface of ASCs, for example, an agent which blocks the expression of integrin, such as, but not limited to, a neutralizing antibody, peptide, protein, aptamer, ribosome, nucleic acid, RNAi, miRNA or small molecule inhibitor of an intergrin expressed on the cell surface of an ASC.

### 1. Ionic Agents

In some embodiments, a modulator of ASC aggregation is heparin. In some embodiments, heparin can be low molecular weight heparin (LMWH) or high molecular weight heparin (HMWH), or modified or heparin-like compounds, such as HEP-PG and HEP-GAG, as disclosed in U.S. Pat. No. 7,314,860, which is incorporated herein in its entirety by reference.

Heparin is a glycosaminoglycan, an acidic mucopolysaccharide composed of D-glucuronic acid and D-glucosamine with a high degree of N-sulphation. It is present in the form of proteoglycan in many mammalian tissues, such as the intestine, liver, lung, being localized in the connective tissue-type mast cells, which line for example the vascular and serosal system of mammals. The main pharmaceutical characteristic of heparin is its ability to enhance the activity of the natural anticoagulant, antithrombin III.

Heparin is generally used as two types; unfractionated or high molecular weight heparin (HMWH) and fractionated or low molecular weight heparin (LMWH). The two types of heparin have the average molecular weight of 15 and 5 kDa, respectively. Most commercial preparations of heparin have a molecular weight of between 4 and 20 kDa depending on their origin, the method of preparation and/or determination.

Heparins exist naturally bound to proteins, forming so called heparin proteoglycans. Usually, the endogenous or native, naturally existing heparin proteoglycans contain 10-15 heparin glycosaminoglycan chains, each chain having a molecular weight in the range of 75±25 kDa, and being bound to one core protein or polypeptide. Each native heparin glycosaminoglycan chain contains several separate heparin units consecutively placed end-to-end, which are cleaved by endoglycosidases in their natural environment. The natural or native conjugates are difficult to prepare in pure form. Thus, they have not been suggested for therapeutical or corresponding use. Heparin glycosaminoglycans (HEP-GAG) belong to a larger group of negatively charged heteropolysaccharides, which generally are associated with proteins forming so called proteoglycans. Examples of other naturally existing glycosaminoglycans are for example chondroitin-4- and 6-sulphates, keratan sulphates, dermatan sulphates, hyaluronic acid, heparan sulphates and heparins. Of said heparin-like compounds existing in nature, only hyaluronic acid is generally not associated with a proteinaceous core molecule.

During the past decades the trend in heparin research has been to develop and use heparin chain units, which have been fractionated for systemic clinical preparations of shorter chains to increase specificity. The generally used two types of standard clinical heparins are the so called unfractionated or high-molecular weight heparins and fractionated or low-molecular-weight heparins. Said two types of heparins have an average molecular weight of 15 and 5 kDa, respectively. In the present invention these two types of heparins are both considered to be lower-molecular-weight heparins. Most commercial preparations have a molecular weight between 4 to 20 kDa depending on their origin, the method of preparation and/or determination. Thus, the commercial heparins belong to the lower-molecular-weight heparins as defined in the present invention.

U.S. Pat. No. 5,529,986, which is incorporated herein in its entirety by reference, discloses synthetic macromolecular heparin conjugate. It consists of at least 20 heparin moieties, but can contain more than 100 heparin moieties, combined with natural or synthetic substantially straight-chained polymer backbones such as polylysine, polyornithine, chitosan, polyamine or polyally 1.

Heparin can also be a low molecular weight (LMWH) of approximately 12 kDa, which is far shorter than the heparin chains in native heparin proteoglycans. The macromolecular heparin molecule described in U.S. Pat. No. 5,529,986 (which is incorporated herein in its entirety by reference), can be used in the admixtures as disclosed herein, which is normally useful as an anticoagulant for coating surfaces of medical devices.

In general, the standard heparin preparations are used for systemic treatment of thrombosis, and as such, are used in the treatment of platelet-poor thrombi, such as venous thrombi, where coagulation activity prevails. Accordingly, heparin is not normally used in repair, or not normally used in conjunction with a cell population such as ASC, or not normally used in cell transplantation methods to prevent aggregation.

Accordingly, in some embodiments, an admixture as disclosed herein can comprise a population of ASC and at least one type of heparin. In some embodiments, heparin used in combination with ASCs can be any one of, or a combination of the following heparin types including, but not limited to, unfractionated heparin (12 kDa) (e.g. HMWH) or low-molecular-weight heparins (LMWH) (7.5 kDa), native heparin proteoglycans (HEP-PG) obtainable from mammalian mast cells, heparin glycosaminoglycan (HEP-GAG), or heparin-like compounds as disclosed in U.S. Pat. No. 7,314,860, which is incorporated herein in its entirety by reference.

In some embodiments, heparin proteoglycans (HEP-PG) or other high molecular weight of the heparin proteoglycans (HEP-PG) based on the multiple structure or the spatial configuration or presentation of its heparin glycosaminoglycan (HEP-GAG) moieties are used in the admixtures as disclosed herein. In some embodiments, admixtures comprise heparin which exists as multiple glycosaminoglycans (HEP-GAG) or heparin proteoglycans (HEP-PG) containing multiple HEP-GAG chains having a size, which mimics the situation in vivo, wherein vascular mast cells were activated and excreted their granules into the external body fluids, wherein the granulate-derived heparin molecules solubilized are used. In some embodiments, admixtures comprise solubilized heparin, where solubilized heparin proteoglycans (HEP-PG) contained in average about 10 heparin glycosaminoglycan (HEP-GAG) moieties, each with a molecular weight of 75±25 kDa. In some embodiments, admixtures comprise heparin which exists by combining several unfractionated or fractionated heparin molecules, herein so called lower-molecular-weight heparin (LMWH) glycosaminoglycan units end-to-end or end-to-side to form multiple glycosaminoglycans (HMWH), either as such or connected to core molecules. In some embodiments, admixtures comprise heparin which exists when multiple amino-sulphated groups of multiple unfractionated heparin chains (12+10 kDa) were coupled with a heterobifunctional coupling reagent, i.e. a spacer or linker molecule, such as N-succinylimidyl-3(2-pyridylthio)propionate (SPSD) to lysine residues present in albumin, a globular protein, offering an optimal core molecules for producing the optimally charged heparin-like compounds of the present invention with the spatial configuration and coupling density.

Thus, in some embodiments, admixtures can comprise heparin-like compounds, which comprise multiple heparin or heparin-like glycosaminoglycan molecules, which have a high molecular weight and consist of several end-to-end and/or end-to-side connected heparin or heparin-like glycosaminoglycan molecules as such or connected to a natural or synthetic, chain-like, preferably short or globular core molecule or lower-molecular-weight heparin or heparin-like glycosaminoglycans conjugated to a globular core molecule. In some embodiments, spacer or linker molecules, which allow attachment of more heparin or heparin-like molecules than the core molecules themselves are used to provide, the desired, sufficient coupling density of said heparin or heparin-like glycosaminoglycan molecules. In some embodiments, where and admixture comprises heparin-like compounds, also have coupling density of negatively charged heparin or heparin-like glycosaminoglycan molecules or units, which provides the heparin-like compounds with a spatial configuration for or closely related to the unique and specific properties of the heparin-like compounds such as HEP-GAG- and HEP-PG-molecules, said property being the capacity of substantially complete inhibition of ASC aggregation.

In some embodiments, the admixture comprises heparin-like compounds which comprise several multiple end-to-end and/or end-to-side connected heparin glycosaminoglycan (HEP-GAG) molecules. Each of which should preferably have a molecular weight of 75±25 kDa or more than 75±25 kDa.

In some embodiment, the admixture comprises heparin-like compounds where multiple heparin or heparin-like glycosaminoglycan molecules can be connected, coupled or conjugated to a natural or synthetic core molecule, which preferably is globular or provides the desired spheroidal configuration, but they can also be connected to more chain-like core molecules.

In some embodiment, the admixture comprises lower-molecular-weight heparin (LMWH) or heparin-like glycosaminoglycan molecules can be connected to core molecules. However, in such cases the core molecule should have a spheroidal or globular configuration. It is also recommendable to use spacer or linker molecules, which allow coupling of much more heparin or heparin-like molecules or units and thus provides a more optimal spatial configuration and a higher coupling density.

The core molecules are advantageously proteins or polypeptides. Useful examples of core molecules are globular proteins, such as albumin, preferably serum albumin of human origin. Another type of core molecules is a polypeptide, which need not be very long and comprises e.g. one or more repetitions of the Ser-Gly-Ser-Gly-sequence. Alternatively, other kinds of amino acid sequences can be used.

In some embodiments, the admixture comprises heparin, or a heparin-like compound both from natural sources, synthetically or semisynthetically or by biotechnological methods, including genetical modifications from commercially available heparins and proteins or polypeptides.

Natural heparin-like compounds are prepared by allowing isolated and purified connective tissue-derived mast cells to grow in a suitable cell culture medium using conditions allowing good cell proliferation and production of heparin-containing granules. After the growth step has been completed, i.e. when the yields of the heparin proteoglycans are optimal, the heparin proteoglycan-containing granules are released by optional activation and/or lysis. The activation step can be carried out with mast cell agonists, which induce mast cell degranulation and release solubilized, multiple HEP-PG-molecules. Preferred agonists are selected from a group consisting of basic polyamines and calcium ionophores. The released granules are allowed to solubilize in the surrounding culture medium and said solubilized heparin proteoglycan (HEP-PG) is collected from the exterior medium. Thereafter, if desired, native multiple heparin glycosaminoglycans (HEP-GAG) can be separated from said heparin-proteoglycans and said heparin glycosaminoglycan (HEP-GAG) molecules can be further coupled to each others in order to obtain heparin glycosaminoglycans with a higher degree of branching and/or multiplicity.

In the synthetic or semisynthetic methods several heparin or heparin-like glycosaminoglycan units can be connected end-to-end and/or end-to-side by covalent bonds. Optionally, especially when using lower-molecular-weight heparin molecules as starting material, coupling reagents, such as spacer or linker molecules, should be used to provide the optional multiplicity and spatial configuration.

The term “heparin-like compound” as used herein means compounds which have a structure resembling that of mast cell-derived heparin proteoglycans and heparin glycosaminoglycans and which are characterized by their capacity of almost complete inhibition of collagen-induced platelet aggregation in flowing whole blood and a coupling density of negatively charged heparin or heparin-like glycosaminoglycan units that gives them the unique properties displayed by the native mast cell-derived heparin proteoglycans (HEP-PG) or heparin glycosaminoglycan (HEP-GAG) molecules obtainable thereof and which property can be measured by the method(s) described in Lassila R, Lindstedt K, Kovanen P T. Arteriosclerosis, Thrombosis, and Vascular Biology 1997; 17 (12): 3578-3587.

The heparin-like compounds useful in the admixtures as disclosed herein, either soluble or immobilized on collagen, are above all characterized by their capacity for inhibiting ASC aggregation.

In its most specific meaning the term “heparin-like compounds” is limited to mast cell-derived heparin proteoglycans (HEP-PG) and heparin glycosaminoglycans (HEP-GAG) obtainable thereof. However, it also includes multiple, unfractionated or fractionated heparin or heparin-like chains coupled to core molecules, either directly or by aid of spacer or linker molecules to provide heparin-like compounds with a unique, spatially optimal configuration, which provides the compounds with a desired, high coupling density of negatively charged heparin or heparin-like glycosaminoglycan molecules or units. The desired high coupling density which provides the unique properties of the heparin-like compounds of the present invention was first found for example in native mast cell-derived heparin proteoglycans (HEP-PG) or the multiple heparin glycosaminoglycan (HEP-GAG) molecules and it seems to explain their properties as well.

The term “heparin or heparin-like proteoglycan” as used herein refers to heparin or heparin-like proteoglycans, which fulfill the prerequisites set up in the definition of heparin-like compounds. Preferably the proteoglycans contain more than three multiple heparin or heparin-like glycosaminoglycan molecules bound to a core molecule. The term “heparin or heparin-like compounds” above all covers native, water-soluble heparin proteoglycans (HEP-PG) obtainable from mammalian connective tissue type mast cells, either by tissue extraction or preferably by cell cultivation. These heparin proteoglycans (HEP-PG) usually comprise approximately 10-15 multiple heparin glycosaminoglycan (HEP-GAG) molecules. Synthetically produced heparin or heparin-like proteoglycans can comprise any number of multiple heparin or heparin-like glycosaminoglycan molecules.

In its broadest aspect the term “heparin-like compounds” means linear or branched heparin-like glycosaminoglycans, i.e. compounds composed of hundreds of monosaccharides comprising amino-groups and being connected or covalently attached to natural or synthetic or semisynthetic core molecules.

Such heparin-like compounds are obtainable from naturally occurring glycosaminoglycan species such as chondriotin sulphates, keratan sulphates, dermatan sulphates, heparan sulphates and/or hyaluronic acid, either as such or modified by chemical or biotechnological means, including recombinant-DNA-techniques to provide molecules, which fulfill the requirements and prerequisites set out above and which are characteristic features of the native mast cell-derivable heparin proteoglycans (HEP-PG) and heparin glycosaminoglycans (HEP-GAG). It is not necessary to isolate the heparin-like glycosaminoglycan molecules from nature. They can be also synthesized or fragments of naturally occurring species can be coupled together especially with heparin-fragments to provide new variants, the properties of which can easily be tested by known methods by those skilled in the art.

One aspect of the present invention relates to the use of heparin, such as low molecular heparin (LMWH), including fractionated and unfractionated heparin, high molecular weight heparin (HMWH), such as HEP-PG and HEP-GAG, for inhibiting aggregation of ASC, as disclosed herein.

Low Molecular Weight Heparin

Compositions containing, procedures for making, and methods for using low molecular weight heparin are described in various patent publications, the contents of which are hereby incorporated by reference, including U.S. Pat. Nos. 4,281,108, 4,687,765, 5,106,734, 4,977,250, 5,576,304, and EP 372 969 (which are incorporated herein in their entirety by reference). Commercially available low molecular weight heparin includes FRAGMINT™ (dalteparin sodium injection, available from Pharmacia, Inc. (Columbus, Ohio)) and LOVENOX™ (enoxaparin sodium injection, available from Rhone-Poulenc Rorer Pharmaceuticals, Inc. (Collegeville, Pa.), described in EP 040 144 (which is incorporated herein in its entirety by reference)).

FRAGMINT™ dalteparin sodium injection is a sterile low molecular weight heparin produced through controlled nitrous acid depolymerization of sodium heparin from porcine intestinal mucosa followed by a chromatographic purification process. It is composed of strongly acidic sulphated polysaccharide chains (oligosaccharide, containing 2,5-anhydro-D-mannitol residues as end groups) with an haverage molecular weight of 5000 and about 90% of the material within the range 2000-9000. It acts by enhancing the inhibition of Factor Xa and thrombin by antithrombin. It is available in a strength of 2500 anti-Factor Xa IU/0.2 mL. FRAGMIN™ is normally used for prophylaxis against deep vein thrombosis, which may lead to pulmonary embolism, in patients undergoing abdominal surgery who are at risk for thromboembolic complications, including those over 40 years of age, obese, undergoing surgery under general anesthesia lasting longer than 30 minutes or who have additional risk factors such as malignancy or a history of deep vein thrombosis or pulmonary embolism. Typically, for patients undergoing abdominal surgery, between 1000 and 5000, e.g. 2500 IU should be administered subcutaneously only, each day, and repeated once each day for 5 to 10 days. Dosage adjustment and routine monitoring of coagulation parameters are not required.

LOVENOX™ enoxaparin sodium injection is a sterile, low molecular weight heparin produced by alkaline degradation of heparin derived from porcine intestinal mucosa. Its structure is characterized by a 2-O-sulfo-4-enepyranosuronic end group at the non-reducing end of the chain. The substance is the sodium salt. The average molecular weight is 4500. LOVENOX™ is used for prevention of deep vein thrombosis, which may lead to pulmonary embolism, following hip or knee replacement surgery. It contains 30 mg enoxaparin sodium in 0.3 mL of Water for Injection, and has an anti-Factor Xa activity of approximately 3000 IU. In patients undergoing hip replacement, or treatment for arterial thrombosis, the recommended dose of LOVENOX™ injection is 30 mg twice daily administered by subcutaneous injection with the initial dose given within 12-24 hours post-operatively provided homeostasis has been established. Treatment should be continued throughout the period of post-operative care until the risk of deep vein thrombosis has been diminished.

In some embodiments, the admixture comprising a population of ASCs and a modulator of ASC aggregation, such as heparin is used in the absence of an antiarrhythmic agents. Antiarrhymic drugs are well known by persons skilled in the art and include, for example, but are not limited to magnesium and amiodarone.

### 2. Chelating Agents

In some embodiments, a modulator of ASC aggregation is a chelating agent such as EDTA. In some embodiments, ethylenediaminetetra-acetic acid (EDTA) can be used in an admixture with ASC at concentrations of about 0.01 to 2.0 mM, preferably at about 0.1 to about 1.0 mM, most preferably at 0.53 mM; at temperatures between 25° to 50° C., preferably between 33° to 40° C., most preferably at 37° C., for periods of between 10 minutes to 3 hours, preferably between 30 minutes to 1 hour, most preferably 45 minutes.

In some embodiments where the modulator of ASC aggregation is EDTA, EDTA can be used at a concentration of between about 0.5 mM-5 mM, for example, at a concentration of at least about 0.5 mM, or at least about 1 mM, or at least about 1.5 mM, or at least about 2.0 mM, or at least about 2.5 mM, or at least about 3.0 mM, or at least about 3.5 mM, or at least about 4.0 mM, or at least about 4.5 mM, or at least about 5.0 mM or any integer between 0.5 mM-5 mM or more than about 5 mM of EDTA. In some embodiments, calcium is added to the admixture, either sequentially after or prior to the addition of EDTA. The concentration of calcium can be, for example between 2 mM-10 mM Ca²⁺, for example, at least about 2 mM, or at least about 3 mM, or at least about 4 mM, or at least about 5 mM, or at least about 6 mM, or at least about 7 mM, or at least about 8 mM, or at least about 9 mM, or at least about 10 mM of Ca²⁺. In some embodiments, the concentration of Ca²⁺ is about 7 mM.

### 3. Proteolytic Agents

In some embodiments, a modulator of ASC aggregation is a proteolytic agent, such as typsin, dispase, collagenase and the like. In some embodiments, collagenase can be used in an admixture with ASC at concentrations between 0.01 to 0.5%, preferably 0.04 to 0.2%, most preferably about 0.1%. In some embodiments, trypsin can be used in an admixture with ASCs as disclosed herein at concentrations between 0.01 to 0.5%, preferably 0.04%, most preferably about 0.2%. In some embodiments, dispase can be used in an admixture with ASCs as disclosed herein at concentrations of 0.5 ng/ml to 10 ng/ml. In some embodiments where the admixture comprises a modulator of ASC aggregation which is a proteolytic enzyme, or functional fragments thereof, the admixture can be incubated at temperatures between 25° to 50° C., or between about 33° to 40° C., or about 37° C., for periods of between 10 minutes to 3 hours, preferably between 30 minutes to 1 hour, most preferably 45 minutes.

4. Agents which Inhibit Expression of Cell Surface Receptors on the ASCs

In some embodiments, a modulator of ASC aggregation is an agent which inhibits the expression of a cell surface receptor or molecule on the ASC which is involved in ASC aggregation. In some embodiments, a modulator of ASC aggregation is an agent which substantially reduces the level of a gene of a cell surface receptor or molecule in the ASC, for example a reduction by at least about 25%, or at least about 50% as compared to the level of the gene of a cell surface receptor or molecule in the ASC in the absence of the agent.

An agent which inhibits the expression or reduces the level of a gene of a cell surface receptor or molecule on the ASC which is involved in ASC aggregation can be any agent, such as but not limited to a neutralizing antibody, nucleic acid, antisense, oligomer, RNAi agent, siRNA, miRNA, aptamer, ribozyme, small molecule inhibitor and the like. In some embodiments, a RNA interference (RNAi) agent useful in the methods as disclosed herein includes, for example but is not limited to antisense nucleotide acid, oligonucleotide, siRNA, shRNA, miRNA, ribozyme, avimirs or variants or derivatives thereof. In some embodiments, a RNAi agent is a shRNA molecule. In such an embodiment, a RNAi molecule would gene silence a cell surface receptor or molecule involved in ASC aggregation, such as for example an integrin.

In some embodiments, an agent which inhibits the expression of, or reduces the level of a gene of a cell surface receptor or molecule on the ASC inhibits the expression of, or reduces the level of a gene encoding one or more integrins expressed on the surface of the ASC. Other targets or target genes for agents which inhibit the expression of, or reduces the level of a gene are any or a combination of genes which encode cell surface receptors or molecules present on the surface of ASCs, and any of the following genes, but are not limited to the following genes and proteins encoding cell surface molecules; CD9; CD13, CD29 (integrin beta 1), CD31, CD34, CD44 (hyaluronate receptor), CD49d,e (integrin alpha 4, 5); CD54 (ICAM1); CD55 (decay accelerating factor); CD63, CD73, CD90, CD105 (endoglin); CD106 (VCAM-1); CD144; CD166 (ALCAM) and HLA-ABC (Class I histocompatibility antigen), as well as aldehyde dehydrogenase and the multidrug-resistance transport protein (ABCG2), VE-cadherin, vascular endothelial growth factor receptor 2, von Willebrand factor.

In one embodiment, a modulator of ASC aggregation is an agent which gene silences at least one gene selected from the group of genes encoding CD9; CD13, CD29 (integrin beta 1), CD31, CD34, CD44 (hyaluronate receptor), CD49d,e (integrin alpha 4, 5); CD54 (ICAM1); CD55 (decay accelerating factor); CD63, CD73, CD90, CD105 (endoglin); CD106 (VCAM-1); CD144; CD166 (ALCAM) and HLA-ABC (Class I histocompatibility antigen), as well as aldehyde dehydrogenase and the multidrug-resistance transport protein (ABCG2), VE-cadherin, vascular endothelial growth factor receptor 2, von Willebrand factor.

In one embodiment, a modulator of ASC aggregation is an agent which gene silences at least one gene selected from the group of genes encoding integrins CD29 (integrin beta 1), CD31, CD34, CD44 (hyaluronate receptor), CD49d,e (integrin alpha 4, 5); CD54 (ICAM1). In some embodiments, a modulator of ASC aggregation is an agent which gene silences the gene encoding the von Willebrand factor.

Inhibition can be achieved by any means. In some embodiments, inhibition is via gene silencing such as for example, using RNAi (RNA interference) methodologies commonly known by persons of ordinary skill in the art or by antisense or ribozyme technology. In some embodiments, the ASC has been genetically modified to express an agent which gene silences a cell surface receptor or molecule on a ASC. By way of an example only, a ASC for use in the admixture can be genetically modified to express an RNAi agent which reduces the expression of one or more of the genes selected from the group of CD9; CD13, CD29 (integrin beta 1), CD31, CD34, CD44 (hyaluronate receptor), CD49d,e (integrin alpha 4, 5); CD54 (ICAM1); CD55 (decay accelerating factor); CD63, CD73, CD90, CD105 (endoglin); CD106 (VCAM-1); CD144; CD166 (ALCAM) and HLA-ABC (Class I histocompatibility antigen), as well as aldehyde dehydrogenase and the multidrug-resistance transport protein (ABCG2), VE-cadherin, vascular endothelial growth factor receptor 2, von Willebrand factor.

In some embodiments, the admixture comprises a ASC which has been genetically modified not to express one or more particular cell surface receptors or molecules on a ASC, for example the gene encoding the cell surface receptor or molecule has been knocked out or deleted or mutated such that the gene is not functional or not expressed at normal levels. In some embodiments, the ASC has been genetically modified whereby a heterologous gene sequence has been introduced such that gene encoding the cell surface receptor or molecule has been modified, e.g. knocked out or deleted or mutated such that the gene is not functional or not expressed at normal levels.

### IV Admixture Compositions of ASCs and Modulators of ASC Aggregation

In some embodiments, a population of ASCs can be combined with at least one modulator of ASC aggregation (e.g. heparin or other modulators as disclosed herein) to form admixtures, compositions for use in the methods such as administration to a subject in the treatment of the subject.

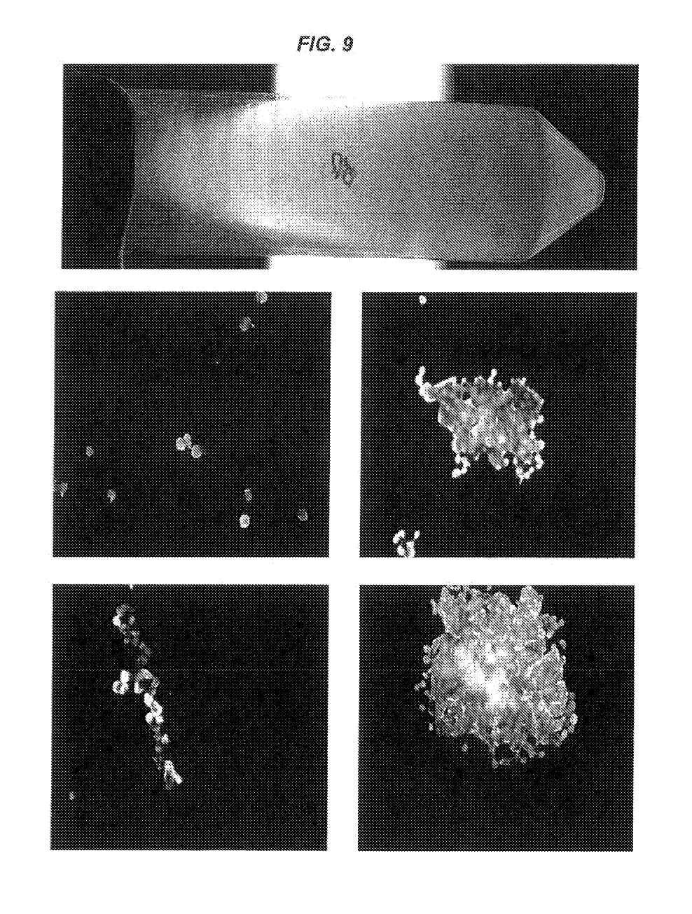

In some embodiments relating to an admixture comprising a combination of a population of ASCs and at least one modulator of ASC aggregation (e.g. heparin or other modulators as disclosed herein), addition to the population of ASC cells, a skilled artisan will appreciate that the effective concentration of ASCs with the modulator of ASC aggregation is dependent on the modulator of ASC aggregation and the intended use of the admixture. Thus, the person of ordinary skill will understand that it is routine to titrate the ASC population in a test or assay to identify the effective concentration to be combined with a certain amount of a particular modulator of ASC aggregation for use in the admixture. For example, to determine the effective concentration of ASCs to be used in an admixture, an admixture can be prepared according the methods as disclosed herein (e.g. see Example 5 and FIGS. 9-13), and/or could be prepared as follows. Eighteen parallel culture preparations comprising 13,000 human ASCs each and either 0, 10, 100, 1000, 10000, or 50,000 units of the modulator to be assessed per/ml are prepared in six triplicate sets. The 18 cultures are assessed and the formation of ASC aggregates or microaggregates quantitatively counted after a defined period of time. To evaluate the effect of each modulator of ASCs on the aggregation of ASCs, similar triplicate cultures could also be incubated, for example in a humidified 37° C., 5% CO₂ incubator, and evaluated over a seven to ten day period.

In some embodiments, the ratio of the population of ASCs to the ratio of a modulator of an ASC aggregation present in an admixture as disclosed herein is dependent on the type and efficacy of the modulator of ASC at reducing ASC aggregates or ASC microaggregates. In some embodiments, where the modulator of ASC aggregation in the admixture is heparin, heparin can be used at a dose of about 50 U/ml of cells, where the cells are at a concentration of about 4−5×10⁷ cells per ml. For example, in some embodiments, and admixture can comprise about 40-100 million ASCs in a total of about 20 ml, also comprising at least about 50 U/ml of heparin.

In some embodiments, the admixture comprises at least about 2×10⁶ cells/ml, or at least about 3×10⁶ cells/ml, or at least about 4×10⁶ cells/ml, or at least about 5×10⁶ cells/ml, or at least about 6×10⁶ cells/ml or any integer between 2×10⁶−6×10⁶ cells/ml. In some embodiments, the admixture comprises less than 2×10⁶ cells/ml.

In some embodiments, the admixture where the modulator of ASC aggregation is heparin, the admixture comprises between 10-100 U/ml of heparin, for example, at least 10 U/ml, or at least about 20 U/ml, or at least about 30 U/ml, or at least about 40 U/ml, or at least about 50 U/ml, or at least about 60 U/ml, or at least about 70 U/ml, or at least about 80 U/ml, or at least about 90 U/ml, or at least about 100 U/ml, or more than 100 U/ml of heparin. In some embodiments, the concentration of heparin is between about 30-60 U/ml, or about 50-100 U/ml or about 100-200 U/ml or about 200-300 U/ml. In some embodiments, the concentration of heparin in a admixture as disclosed herein is about 40 U/ml, or about 45 U/ml, or about 50 U/ml, or about 55 U/ml, or about 60 U/ml.

In some embodiments, the admixture where the modulator of ASC aggregation is heparin, the admixture comprises between 25 μg/ml-1000 μg/ml of heparin, for example, at least 10 μg/ml, or at least about 20 μg/ml, or at least about 25 μg/ml, or at least about 30 μg/ml, or at least about 40 μg/ml, or at least about 50 μg/ml, or at least about 75 μg/ml, or at least about 100 μg/ml, or at least about 150 μg/ml, or at least about 200 μg/ml, or at least about 250 μg/ml, or at least about 300 μg/ml, or at least about 350 μg/ml, or at least about 400 μg/ml, or at least about 450 μg/ml, or at least about 500 μg/ml, or any integer between 25 μg/ml and 500 μg/ml, or more than 500 μg/ml of heparin. In some embodiments, the admixture comprises heparin as the modulator of ASC aggregation, where the concentration of heparin in the admixture comprises at least about 400 μg/ml-1000 μg/ml of heparin, for example, at least about or at least about 400 μg/ml, or at least about 500 μg/ml, or at least about 600 μg/ml, or at least about 700 μg/ml, or at least about 800 μg/ml, or at least about 900 μg/ml, or at least about 1000 μg/ml, or any integer between 400 μg/ml and 1000 μg/ml, or more than 1000 μg/ml of heparin. In some embodiments, the concentration of heparin is between about 25-400 μg/ml, or about 50-100 μg/ml or about 100-200 μg/ml or about 200-300 μg/ml or about 300-400 μg/ml or about 400-500 μg/ml of heparin. In some embodiments, the concentration of heparin in a admixture as disclosed herein is about 40 μg/ml, or about 45 μg/ml, or about 50 μg/ml, or about 55 μg/ml, or about 60 μg/ml of heparin.

In one embodiment, a population of ASC and at least one modulator of ASC aggregation cells are administered substantially concurrently. For example, in one embodiment, a population of ASC is combined with at least one modulator of ASC aggregation cells immediately prior to administration into the subject. In another embodiment, a population of ASC is administered separately to the subject from the administration of at least one modulator of ASC aggregation. Optionally, where the population of ASCs are administered separately from the administration of at least one modulator of ASC aggregation, there is a temporal separation in the administration of the ASCs and the modulators of ASC aggregation. The temporal separation may range from less than about 10 seconds, or less than about 30 seconds, to less than about a minute in time, to less than about an hour or days in time. The determination of the optimal timing and order of administration is readily and routinely determined by one of ordinary skill in the art. In some embodiments, the temporal separation is typically less than about 30 second, or less than about 20 second or less than about 10 seconds. In some embodiments for example, where the modulator of ASC is heparin, a population of ASC can be administered prior to, or following a bolus of heparin, such as unfractionated heparin as disclosed herein, where the temporal separation between the administration of the heparin and the population of ASCs is less than 1 hr, or less than 30 mins or less than 10 mins, or less than about 5 mins, or less than about 1 min or less than about 30 second or less than about 10 second. In such embodiments, a dose of the bolus of heparin can be within the normal range for administration, for example about 300 U/kg, or more than about 300 U/kg, for example 300-500 U/kg, or 300-1000 U/kg or more than 1000 U/kg, or less than 300 U/kg, such as between 100-300 U/kg.

In all embodiments, at least one or more modulators of ASC aggregation can be added to a population of ASC at any appropriate time in the preparation of the ASC population. A skilled artisan will appreciate that one or more modulators of ASC aggregation can be added during the preparation (e.g. during washing steps and/or isolation steps) of the ASC population, as well as at the final resuspension of the isolated population of ASCs. Accordingly, in some embodiments, one or more modulators of ASC aggregation can be present in a wash buffer and/or a resuspension buffer, where the wash buffer and/or resuspension buffer is then added to a population of ASC. In some embodiments, a wash buffer which is used for washing a population of ASCs can comprises one or more modulators of ASC aggregation. In some embodiments, a resuspension buffer which is used for resuspending a population of ASC comprises one or modulators of ASC aggregation. In some embodiments, a cryopreservation buffer can comprise one or more modulators of ASC aggregation.

As discussed herein, a device which is used to harvest and isolate a population of ASC can be configured (e.g. by modifications to hardware and/or software) to add at least one modulator of ASC to a population of ASCs, either at one or more points during the isolation and washing procedure, and/or at the final resuspension of the ASC population in a resuspension buffer. In some embodiments, the amount of a modulator of ASC to be titratated to determine the amount of at least one modulator of ASC to be added to a population of ASCs, depending on, for example, (i) the type of modulator of ASC aggregation (e.g. heparin versus other types of modulators of ASC aggregation), (ii) the concentration of modulator of ASC aggregation and (iii) the timepoint in the isolation/washing procedure or at the end of the procedure when the modulator of ASC aggregation is added.

In some embodiments, a population of ASC is incubated with at least one modulator of ASC aggregation for an appropriate amount of time to reduce or inhibit the formation of ASC aggregates. In some embodiments, the amount is for at least about 30 mins, or at least about 45 mins, or at least about 60 mins, or at least about 75 mins, or at least about 90 mins, or at least about 120 mins, or at least about 135 mins, or at least about 150 mins, or more than about 150 mins.

In some embodiments, an admixture comprising a combination of a population of ASCs and at least one modulator of ASC aggregation (e.g. heparin or other modulators as disclosed herein) can further comprise additional cells, such as cells termed “relevant cells” or other stem cells as disclosed herein.

The skilled artisan will understand that further refinement of the appropriate number of additional cells for admixture comprising ASC cells and a modulator of ASC aggregation can be determined by additional experiments, based on the results of the above procedure. For example, if in the first experiment that 1000 additional cells/ml demonstrated the best results, additional tests using 500, 2000 and 6000 cells/ml would allow further refinement of the optimal number of additional cells per admixture comprising ASCs and a modulator of ASC aggregation. A similar procedure could be followed to determine the appropriate concentration of an additional cell type, such as other relevant Cells, genetically engineered cells, or combinations thereof, in a admixture comprising ASCs and a modulator of ASC aggregation.

### V. Use of the Admixture

In some embodiments, the admixture as disclosed herein can be administered into a subject in order to treat a subject. Administration of the admixture can occur in a wide variety of ways. Preferred modes of administration are parenteral, intraperitoneal, intravenous, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump, or via catheter. In one embodiment, the admixture can be administered in a slow release formulation such as a direct tissue injection or bolus, implant, microparticle, microsphere, nanoparticle ornanosphere. In some embodiments, the admixture as disclosed herein is administered to a subject via intravascular delivery.

In some embodiments, the admixture is administered to a subject via intracoronary administration. In some embodiments, the admixture as disclosed herein is used for the treatment of cardiovascular conditions, such as those intracoronary administration for the treatment of heart failure or myocardial infarction. In some embodiments, the admixture can be used in methods as disclosed in International Patent Application WO2006/0127007, which is incorporated herein in its entirety by reference.

The cells described herein can be used in combination with any known technique of tissue engineering, including but not limited to those technologies described in patents and publications, e.g. U.S. Pat. Nos. 5,902,741 and 5,863,531 to Advanced Tissue Sciences, Inc.) as well as, but not limited to: U.S. Pat. No. 6,139,574, Vacanti et al. (Oct. 31, 2000) Vascularized Tissue Regeneration Matrices Formed By Solid Free Form Fabrication Techniques;U.S. Pat. No. 5,759,830, Vacanti et al. (Jun. 2, 1998) Three-Dimensional Fibrous Scaffold Containing Attached Cells For Producing Vascularized Tissue In Vivo; U.S. Pat. No. 5,741,685, Vacanti, (Apr. 21, 1998) Parenchymal Cells Packaged Inlnmunoprotective Tissue For Implantation; U.S. Pat. No. 5,736,372, Vacanti et al. (Apr. 7, 1998) Biodegradable Synthetic Polymeric Fibrous Matrix Containing Chondrocyte For In Vivo Production Of A Cartilaginous Structure; U.S. Pat. No. 5,804,178, Vacanti et al. (Sep. 8, 1998) Implantation Of Cell-Matrix Structure Adjacent Mesentery, Omentum Or Peritoneum Tissue; U.S. Pat. No. 5,770,417, Vacanti et al. (Jun. 23, 1998) Three-Dimensional Fibrous Scaffold Containing Attached Cells For Producing Vascularized Tissue In Vivo; U.S. Pat. No. 5,770,193, Vacanti et al. (Jun. 23, 1998) Preparation of Three-Dimensional Fibrous Scaffold For Attaching Cells To Produce Vascularized Tissue In Vivo; U.S. Pat. No. 5,709,854, Griffith-Cima et al. (Jan. 20, 1998) Tissue Formation By Injecting A Cell-Polymeric Solution That Gels In Vivo; U.S. Pat. No. 5,516,532, Atala et al. (May 14, 1998) Injectable Non-Immunogenic Cartilage And Bone Preparation; U.S. Pat. No. 5,855,610, Vacanti et al. (Jan. 5, 1999) Engineering Of Strong, Pliable Tissues; U.S. Pat. No. 5,041,138, Vacanti et al. (Aug. 20, 1991) Neomorphogenesis Of Cartilage In Vivo From Cell Culture; U.S. Pat. No. 6,027,744, Vacanti et al. (Feb. 22, 1900) Guided Development and Support Of Hydrogel-Cell Compositions; U.S. Pat. No. 6,123,727, Vacanti et al. (Sep. 26, 2000) Tissue Engineered Tendons And Ligament; U.S. Pat. No. 5,536,656, Kemp et al. (Jul. 16, 1996) Preparation Of Tissue Equivalents By Contraction Of A Collagen Gel Layered On A Collagen Gel; U.S. Pat. No. 5,144,016, Skjak-Braek et al. (Sep. 1, 1992) Alginate Gels; U.S. Pat. No. 5,944,754, Vacanti (Aug. 31, 1999) Tissue Re-Surfacing With Hydrogel-Cell Compositions; U.S. Pat. No. 5,723,331, Tubo et al. (Mar. 3, 1998) Methods And Compositions For The Repair Of Articular Cartilage Defects In Mammals; U.S. Pat. No. 6,143,501, Sittinger et al. (Nov. 7, 2000) Artificial Tissues, Methods For The Production And The Use Thereof. (The patents and patent applications listed in the above-paragraph are incorporated herein in their entirety by reference).

The presence of the ASCs of the invention may be detected in a subject by a variety of techniques including, but not limited to, flow cytometirc, immunohistochemical, in situ hybridization, and/or other histologic or cellularbiologic techniques. See, for example, Kopen et al., (1999) Proc Natl Acad Sci 96:10711-10716.

Disorders that can be treated by infusion of the admixture as disclosed herein include, but are not limited to, diseases resulting from cardiac dysfunction, including heart failure and myocardial infarction.

The invention further provides for a method of treating an injured tissue in an individual comprising: (a) determining a site of tissue injury in the individual; and (b) administering the admixture of the invention into and around the site of tissue injury. In one embodiment, the tissue is cardiac muscle. In one embodiment, the ASC present in the admixture are derived from an autologous source. In a further embodiment, the tissue injury is a myocardial infarction, cardiomyopathy or congenital heart disease.

In one embodiment of the above methods, the subject is a human and the ASCs are human cells. In alternative embodiments, the admixture as disclosed herein can be used to treat a circulatory disorder, such as one selected from the group consisting of cardiomyopathy, myocardial infarction, and congenital heart disease. In some embodiments, the circulatory disorder is a myocardial infarction. In some embodiments the admixture can be intracoronary administered into a cardiac muscle for the treatment of myocardial infarction, and can reduce the size of the myocardial infarct. It is also contemplated that the admixture as disclose herein administered via intracoronary delivery can be used to treat myocardial infarction by reducing the size of the scar resulting from the myocardial infarct. In some embodiments, the admixture as disclosed herein can be administered directly to heart tissue of a subject, or is administered systemically.

In some embodiments, the admixture as disclosed herein can be used in a method of treating circulatory damage in the heart or peripheral vasculature which occurs as a consequence of genetic defect, physical injury, environmental insult or damage from a stroke, heart attack or cardiovascular disease (most often due to ischemia) in a subject, the method comprising administering (including transplanting), the admixture comprising effective number or amount of ASCs to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

In some embodiments, the effects administration of the admixture as disclosed herein can be demonstrated by the methods disclosed in the examples or by alternative methods, including but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of administration of the admixture to a subject can be evident over the course of days to weeks after the procedure. However, beneficial effects may be observed as early as several hours after the procedure, and may persist for several years.

In some embodiments, the admixture can be used for the treatment of cancer, as disclosed in U.S. patent application with U.S. application Ser. No. 12/511,940, filed on Jul. 29, 2009 which is incorporated herein in its entirety by reference. In some embodiments, the admixture as disclosed herein can be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering the admixture as disclosed herein for reconstituting cardiac function directly to the chambers of the heart, the pericardium, or the interior of the cardiac muscle at the desired location. In some embodiments, the admixture can be administered to a recipient heart by intracoronary injection, e.g. into the coronary circulation. In alternative embodiments, the admixture can be administered by intramuscular injection into the wall of the heart.

In another embodiment, the adipose-derived stromal cells present in the admixture can be genetically modified, e.g., to express exogenous genes or to repress the expression of endogenous genes. In accordance with this embodiment, the cell is exposed to a gene transfer vector comprising a nucleic acid including a transgene, such that the nucleic acid is introduced into the cell under conditions appropriate for the transgene to be expressed within the cell. The transgene generally is an expression cassette, including a coding polynucleotide operably linked to a suitable promoter. The coding polynucleotide can encode a protein, or it can encode biologically active RNA, such as antisense RNA (RNAi) or a ribozyme. Thus, the coding polynucleotide can encode a gene conferring, for example, resistance to a toxin, a hormone (such as peptide growth hormones, hormone releasing factor, sex hormones, adrenocorticotrophic hormones, cytokines such as interferons, interleukins, and lymphokines), a cell surface-bound intracellular signaling moiety such as cell-adhesion molecules and hormone receptors, and factors promoting a given lineage of differentiation, or any other transgene with known sequence.

The expression cassette containing the transgene should be incorporated into the genetic vector suitable for delivering the transgene to the cell. Depending on the desired end application, any such vector can be so employed to genetically modify the cells (e.g., plasmids, naked DNA, viruses such as adenovirus, adeno-associated virus, herpesvirus, lentivirus, papillomavirus, retroviruses, etc.). Any method of constructing the desired expression cassette within such vectors can be employed, many of which are well known in the art, such as by direct cloning, homologous recombination, etc. The desired vector will largely determine the method used to introduce the vector into the cells, which are generally known in the art. Suitable techniques include protoplast fusion, calcium-phosphate precipitation, gene gun, electroporation, and infection with viral vectors.

### V. Genetically Modified ASCs for Use in an Admixture

In some embodiments, ASCs used in the methods, compositions and admixtures as disclosed herein are genetically modified. Methods to genetically engineer ASC cells are disclosed in U.S. application Ser. No. 12/511,940, filed on Jul. 29, 2009 which is incorporated herein in its entirety by reference.

Human ASCs can be genetically engineered to generate cells that constitutively express human gamma interferon (γ-IFN), essentially as described by Stopeck et al., Cell Transplantation 6:18, 1997. The human SVF cells can be resuspended in M199 supplemented with 20% heat-inactivated FBS, 5 mM HEPES, 1.7 mM L-glutamine, and 60 .mu.g/mL endothelial cell growth supplement (Jarrell et al., J. Vasc. Surg. 1:757 64, 1984) containing 25 .mu.g/mL heparin and plated on gelatin coated polystyrene T-25 tissue culture flasks and incubated in a conventional humidified 37° C., 5% CO₂ incubator and maintained in culture.

Supernatants of high titer (1×10⁶−1×10⁷ cfu/mL) recombinant retrovirus containing either the E. coli beta-galactosidase (β-gal) or human γ-IFN gene were obtained from Viagene, Inc. (San Diego, Calif.). These recombinant retroviruses comprise a Moloney murine leukemia virus genome with viral structural genes replaced by either the β-gal or the human γ-IFN gene. T-25 flasks of human endothelial cells at 30 40% confluency are transduced for 6 18 hours on 2 consecutive days with media containing 750 μg/mL protamine sulfate and retrovirus supernatants at a multiplicity of infection of 5.

Forty-eight hours after transduction, cells are fixed with 2% formaldehyde prior to staining with X-gal solution (5 mM potassium ferricyanide, 5 mM potassium ferrocyanide, 2 mM MgCl₂, and 1 mg/,L X-gal (Sigma, St. Louis, Mo.) in PBS overnight at 37° C. The transduction efficiency is calculated as the number of cells staining positive for .beta.-gal divided by the total number of cells counted. β-gal transduced or human y-IFN transduced endothelial cells are selected using 1 mg/mL G418 (Gibco BRL) selection medium.

Total RNA is extracted from transduced and control endothelial cells using Trizol (Gibco BRL) for RT-PCR analysis, as described. Human endothelial cells transduced according to this procedure reportedly produce 80-130 pg/mL of human γ-IFN per 10⁵ cells after 24 hours in culture (see Stopeck et al., Cell Transplantation 6:18, 1997 and U.S. Pat. No. 5,957,972 (which is incorporated herein in its entirety by reference).

The skilled artisan will understand that replacement of the human γ-IFN or β-gal gene in these recombinant retrovirus vectors with alternate genes of interest requires only routine manipulation using techniques generally known in the art. Thus, any number of genes of interest may be transduced into and expressed by endothelial cells following this exemplary technique. The skilled artisan will also understand that, following techniques generally known in the art, a variety of mammalian cells can routinely be transduced or transfected to express virtually any gene product of interest (see, e.g., Twyman, Advanced Molecular Biology: A Concise Reference, Bios Scientific Publishers, Springer Verlag New York, particularly Chapter 24). Particularly useful gene products of interest include, for example, but without limitation, cytokines, insulin, human growth hormone, plasminogen activator, soluble CD4, Factor VIII, Factor IX, von Willebrand Factor, urokinase, hirudin, interferons, including α-, β- and γ-interferon, tumor necrosis factor, interleukins, hematopoietic growth factor, antibodies, glucocerebrosidase, adenosine deaminase, phenylalanine hydroxylase, human growth hormone, insulin, erythropoietin, VEGF, angiopoietin, hepatocyte growth factor, PLGF, and other proteins or gene products appropriate for local or systemic delivery, particularly blood-borne delivery.

Genetically engineered cells, particularly genetically engineered ASCs, may be incorporated into the admixture at appropriate concentrations, as described. The skilled artisan that a wide variety of techniques may be used to genetically modify cells, i.e., transferring genes and nucleic acids of interest into recipient cells, using techniques generally known in the art, including, but not limited to: transfection (e.g., the uptake of naked nucleic acid), for example, but not limited to polyethylene glycol transfection, chemical transfection (e.g., using calcium phosphate and DEAE dextran), lipofection, electroporation, direct injection, and microballistics; and transduction, using a number of viral vectors, such as, without limitation, adenovirus vectors, herpesvirus vectors, retrovirus vectors, including, but not limited to lentivirus vectors. Descriptions of such techniques may be found in, among other places, Ausubel et al., Current Protocols in Molecular Biology (including supplements through March 2002), John Wiley & Sons, New York, N.Y., 1989; Sambrook et al., Molecular Cloning: A Laboratory Manual, 2nd Ed., Cold Spring Harbor Laboratory Press, Cold Spring Harbor, N.Y., 1989; Sambrook and Russell, Molecular Cloning: A Laboratory Manual, 3rdEd., Cold Spring Harbor Laboratory Press, Cold Spring Harbor, N.Y., 2001; Beaucage et al., Current Protocols in Nucleic Acid Chemistry, John Wiley & Sons, New York, N.Y., 2000 (including supplements through March 2002); Short Protocols in Molecular Biology, 4th Ed., Ausbel, Brent, and Moore, eds., John Wiley & Sons, New York, N.Y., 1999; Davis et al., Basic Methods in Molecular Biology, McGraw Hill Professional Publishing, 1995; Molecular Biology Protocols (see the highveld.com website), Protocol Online (protocol-online.net); and Twyman, Advanced Molecular Biology: A Concise Reference, Bios Scientific Publishers, Springer-Verlag New York.

### VI. Pharmaceutical Compositions

Pharmaceutical compositions comprising an admixture comprising a population of ASC and at least one modulator of ASC aggregation are also contemplated by the present invention. These compositions comprise an effective number of ASCs and an effective amount of a modulator of ASC aggregation, optionally, in combination with a pharmaceutically acceptable carrier, additive or excipient.

In certain aspects of the present invention, the admixture comprising a population of ASC and at least one modulator of ASC aggregation cells are administered to the subject in need of a transplant in sterile saline. In other aspects of the present invention, an admixture comprising a population of ASC and at least one modulator of ASC aggregation cells are administered in Hanks Balanced Salt Solution (HBSS) or Isolyte S, pH 7.4. Other approaches may also be used, including the use of serum free cellular media. In one embodiment, the cells are administered in plasma or fetal bovine serum, and DMSO. Systemic administration of the admixture comprising a population of ASC and at least one modulator of ASC aggregation cells to the subject may be preferred in certain indications, whereas direct administration at the site of or in proximity to the diseased may be preferred in certain diseases or conditions, such as cardiac conditions and the like.

The admixture comprising a population of ASC and at least one modulator of ASC aggregation cells can optionally be packaged in a suitable container with written instructions for a desired purpose, such as the reconstitution of admixture to administer to a subject.

In some embodiment, admixture comprising a population of ASC and at least one modulator of ASC aggregation cells are administered with an additional agent, for example, an anticoagulation agent. In one embodiment, an admixture comprising a population of ASC and at least one modulator of ASC aggregation cells are combined with the anticoagulation agent to administration into the subject. In another embodiment, the admixture comprising a population of ASC and at least one modulator of ASC aggregation cells are administered separately to the subject from the anticoagulation agent, as disclosed herein in the Examples. Such anticoagulation agent can be for example but is not limited to, unfractionated heparin, as disclosed herein, where a bolus dose of unfractionated heparin can be administered by any suitable route such as I.V. administration within the normal range for administration, for example about 300 U/kg, or more than about 300 U/kg, for example 300-500 U/kg, or 300-1000 U/kg or more than 1000 U/kg, or less than 300 U/kg, such as between 100-300 U/kg. Optionally, where the admixture is administered separately from the anticoagulation agent, there is a temporal separation in the administration of the admixture and the anticoagulation agent. The temporal separation may range from about less than a minute in time, to about hours or days in time. The determination of the optimal timing and order of administration is readily and routinely determined by one of ordinary skill in the art.

Other anticoagulation agents can also be administered substantially concurrently with (e.g. at the same time), substantially subsequently to, or substantially prior to, the administration of the admixture as disclosed herein. In some embodiments, such anticoagulation agents include but are not limited to, low molecular weight heparins, unfractionated heparins or heparin-like compounds as disclosed herein, Dalteparin (FRAGMIN®); Danaparoid (ORGARAN®); Enoxaparin (LOVENOX®); Heparin (HEP-LOCK, HEP-PAK, HEP-PAK CVC, HEPARIN LOCK FLUSH); Tinzaparin (INNOHEP®); Warfarin (COUMADIN®); GPIIb/IIIa receptor antagonists and Antagonists for glycoprotein IIb/IIIb fibrinogen receptor as disclosed in U.S. Pat. No. 6,136,794 which is incorporated herein in its entirety by reference.

Anticoagulant drugs, also called anticlotting drugs or blood thinners, which can be administered with an admixture as disclosed herein include inhibitors of clotting factor synthesis, such as anticoagulants inhibit the production of certain clotting factors in the liver, for example warfarin (COUMADIN®). Anticoagulant agents which can be administered with an admixture as disclosed herein include inhibitors of thrombin. Thrombin inhibitors interfere with blood clotting by blocking the activity of thrombin, such as heparin, lepirudin (REFLUDAN®). Anticoagulant agents which can be administered with an admixture as disclosed herein include antiplatelet drugs. Antiplatelet drugs interact with platelets, which is a type of blood cell, to block platelets from aggregating into harmful clots, such as aspirin, ticlopidine (TICLID®), clopidogrel (PLAVIX®), tirofiban (AGGRASTAT®), and eptifibatide (INTEGRILIN®).

In one embodiment, admixture comprising a population of ASC and at least one modulator of ASC aggregation cells are administered with a differentiation agent. In one embodiment, an admixture comprising a population of ASC and at least one modulator of ASC aggregation cells are combined with the differentiation agent to administration into the subject. In another embodiment, the admixture comprising a population of ASC and at least one modulator of ASC aggregation cells are administered separately to the subject from the differentiation agent. Optionally, admixture is administered separately from the differentiation agent, there is a temporal separation in the administration of the admixture and the differentiation agent. The temporal separation may range from about less than a minute in time, to about hours or days in time. The determination of the optimal timing and order of administration is readily and routinely determined by one of ordinary skill in the art.

An admixture comprising a population of ASC and at least one modulator of ASC aggregation can be administered alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. The ASC population present in an admixture comprising a population of ASC and at least one modulator of ASC aggregation, can be modified by insertion of DNA or by placement in cell culture in such a way as to change, enhance, or supplement the function of the ASCs for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998). In another aspect, the ASC present in the admixture can be optionally transduced with one or more nucleic acid sequences encoding an agent which gene silences a cell surface molecule or receptor involved in ASC aggregation. In some embodiments, the ASC present in the admixture can be optionally transduced with a gene encoding a pro-angiogenic growth factor(s).

In some embodiments, an admixture as disclosed herein can include additional agents, such as but not limited to growth factors, cytokines, hormones, angiogenic factors, immunosuppressive drugs and the like. In some embodiments, the admixture does not comprise or is not administered in combination with an antiarrhythmic agents, for example, but not limited to magnesium and amiodarone.

By “growth factors, cytokines, hormones” is intended the following specific factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogeneticprotein at concentrations of between pigogram/ml to milligram/ml levels. At such concentrations, the growth factors, cytokines and hormones useful in the methods of the invention are able to induce, up to 100% the formation of blood cells (lymphoid, erythroid, myeloid or platelet lineages) from adipose derived stromal cells in colony forming unit (CFU) assays. (Moore et al. (1973) J. Natl. Cancer Inst. 50:603 623; Lee et al. (1989) J. Immunol. 142:3875 3883; Medina et al. (2993) J. Exp. Med. 178:1507 1515.

It is further recognized that additional components may be added to the admixture. Such components may be antibiotics, antimycotics, albumin, amino acids, and other components known to the art for the culture of cells. Additionally, components may be added to enhance the differentiation process of ASCs.

The term “chemical agents” is meant to include, but not be limited to, antioxidant compounds such as butylated hydroxyanisole (BHA) or 2-mercaptoethanol, steroids, retinoids, and other chemical compounds or agents that induce the differentiation of adipose derived stromal cells.

Practice of the present invention will employ, unless indicated otherwise, conventional techniques of cell biology, cell culture, molecular biology, microbiology, recombinant DNA, protein chemistry, and immunology, which are within the skill of the art. Such techniques are described in the literature. See, for example, Molecular Cloning: A Laboratory Manual, 2nd edition. (Sambrook, Fritsch and Maniatis, eds.), Cold Spring Harbor Laboratory Press, 1989; DNA Cloning, Volumes I and II (D. N. Glover, ed), 1985; Oligonucleotide Synthesis, (M. J. Gait, ed.), 1984; U.S. Pat. No. 4,683,195 (Mullis et al.,); Nucleic Acid Hybridization (B. D. Hames and S. J. Higgins, eds.), 1984; Transcription and Translation (B. D. Hames and S. J. Higgins, eds.), 1984; Culture of Animal Cells (R. I. Freshney, ed). Alan R. Liss, Inc., 1987; Immobilized Cells and Enzymes, IRL Press, 1986; A Practical Guide to Molecular Cloning (B. Perbal), 1984; Methods in Enzymology, Volumes 154 and 155 (Wu et al., eds), Academic Press, New York; Gene Transfer Vectors for Mammalian Cells (J. H. Miller and M. P. Calos, eds.), 1987, Cold Spring Harbor Laboratory; Immunochemical Methods in Cell and Molecular Biology (Mayer and Walker, eds.), Academic Press, London, 1987; Handbook of Experiment Immunology, Volumes I-IV (D. M. Weir and C. C. Blackwell, eds.), 1986; Manipulating the Mouse Embryo, Cold Spring Harbor Laboratory Press, 1986. (The patents listed in this paragraph are incorporated herein in their entirety by reference).

Some embodiments of the present invention may be defined in any of the following numbered paragraphs:
1. An admixture comprising a combination of an isolated population of adipose-derived stromal cells (ASCs) and a modulator of ASC aggregation.
2. The admixture of paragraph 1, wherein the modulator of ASC aggregation inhibits aggregation of the ASCs.
3. The admixture of paragraph 1, wherein the modulator of ASC aggregation is an ionic agent.
4. The admixture of paragraph 3, wherein the ionic agent is heparin or an analogue thereof.
5. The admixture of paragraph 1, wherein the modulator of ASC aggregation is a chelating agent.
6. The admixture of paragraph 5, wherein the chelating agent is EDTA or an analogue thereof.
7. The admixture of paragraph 1, wherein the modulator of ASC aggregation is a proteolytic agent.
8. The admixture of paragraph 7, wherein the proteolytic agent is trypsin or dispase or an analogue thereof.
9. The admixture of paragraph 1, wherein the modulator of ASC aggregation n is an agent which blocks the expression of an attachment protein expressed on the cell surface of ASCs.
10. The admixture of paragraph 9, wherein the modulator of ASC aggregation is an agent which blocks the expression of integrin.
11. The admixture of paragraph 1, wherein the ASC are human.
12. The admixture of paragraph 1, wherein the ASC are harvested from a human subject.
13. The admixture of paragraph 1, wherein the ASCs are freshly isolated ASC.
14. The admixture of paragraph 1, wherein the ASC are cultured ASC.
15. The admixture of paragraph 1, wherein the ASC are genetically modified ASC.
16. The admixture of paragraph 1, wherein the ASC in combination with a modulator of ASC is cryopreserved.
17. A method of inhibiting formation of macromolecular aggregates of adipose-derived stromal cells (ASC), comprising contacting a population of ASC with a modulator of ASC aggregation.
18. The method of paragraph 17, wherein the macromolecular aggregate of ASC comprises at least 4 adipose-derived stromal cells.
19. The method of paragraph 17, wherein the modulator of ASC aggregation inhibits aggregation of the ASCs.
20. The method of paragraph 17, wherein the modulator of ASC aggregation is an ionic agent.
21. The method of paragraph 20, wherein the ionic agent is heparin or an analogue thereof.
22. The method of paragraph 17, wherein the modulator of ASC aggregation is a chelating agent.
23. The method of paragraph 22, wherein the chelating agent is EDTA or an analogue thereof.
24. The method of paragraph 17, wherein the modulator of ASC aggregation is a proteolytic agent.
25. The method of paragraph 24, wherein the proteolytic agent is trypsin or dispase or an analogue thereof.
26. The method of paragraph 17, wherein the modulator of ASC aggregation n is an agent which blocks the expression of an attachment protein expressed on the cell surface of ASCs.
27. The method of paragraph 26, wherein the modulator of ASC aggregation is an agent which blocks the expression of integrin.
28. The method of paragraph 17, wherein the ASC are human.
29. The method of paragraph 17, wherein the ASC are harvested from a human subject.
30. The method of paragraph 17, wherein the ASCs are freshly isolated ASC.
31. The method of paragraph 17, wherein the ASC are cultured ASC.
32. The method of paragraph 17, wherein the ASC are genetically modified ASC.
33. The method of paragraph 17, wherein the ASC in combination with a modulator of ASC is cryopreserved.
34. Use of the admixture of any of paragraphs 1-16 for administering to a subject in need of a transplant of adipose-derived stromal cells (ASC).
35. The use of the admixture of paragraph 34, wherein the subject is suffering with insufficient cardiac function or disease or disorder.
36. The use of the admixture of paragraph 35, wherein the disease or disorder is heart failure.
37. The use of the admixture of paragraph 35, wherein the disease or disorder is myocardial infarction.
38. The use of the admixture of paragraph 34, wherein the admixture comprises one or more additional agents, or any combination thereof selected from the group consisting of: angiogenic factors, growth factors, and immunosuppressive drugs.
39. The use of the admixture of paragraph 34, wherein administration is via endomyocardial, epimyocardial, intraventricular, intracoronary, retrograde coronary sinus, intra-arterial, intra-pericardial or intravenous administration.
40. The use of the admixture of paragraph 34, wherein the subject is human.
41. The use of the admixture of paragraph 40, wherein the ASC in the admixture are harvested from the same subject to which the admixture is administered to.
42. A use of heparin for inhibiting aggregation of freshly isolated ASC.
43. A method of reducing the consequences of a myocardial infarction in a subject, the method comprising the steps of:
   a. freshly isolating ASC from the subject;
   b. forming a mixture of the freshly isolated ASC and heparin; and
   c. delivering via intracoronary catheter the mixture to post-infarction myocardium of the subject.
44. wherein the delivery of the mixture of freshly isolated ASC and heparin reduce the consequences of a myocardial infarction in the subject.

Although the invention has been described with reference to various applications, methods, and compositions, it will be appreciated that various changes and modifications may be made without departing from the invention. The foregoing examples are provided to better illustrate the invention and are not intended to limit the scope of the invention.

### EXAMPLES

The examples presented herein relate to the methods and compositions comprising a combination of ASC and an agent which inhibits aggregation of ASC, such as an ionic agent, (e.g. heparin or heparin-like compounds), chelating agents, (e.g. EDTA), proteolytic agents (e.g. trypsin or dispase) and agents which block the expression of cell-surface receptors on ASC (e.g. inhibition of expression of integrins) and the like. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

A target dose of 40×10⁶ ASC, either in medium only (ASC, n=5), medium with 50 U/ml heparin (ASC—H, n=4); or vehicle alone (n=6) was delivered to the LAD of normal pigs. Electrocardiograms were continuously recorded in ambulatory animals. Troponin-I, TIMI frame counts, ventriculography, and ⁹⁹ Tc-sestamibi SPECT imaging were evaluated following delivery and at sacrifice (7 days), along with histology. No pathologies were noted in the vehicle group. In the ASC group, small infarctions were noted histologically and by elevated troponin-I (4.0±4.8 ng/ml). Consecutive ventricular rhythm (cVR) occurred in 4 of 5 ASC pigs, ranging from 6.4±1.4 to 28.9±5.6 hours post-delivery. The proportion of cVR duration over 21 hours was 16.4±15.3, with a cycle length of 577±86 msec. In the ASC-H group, troponin-I elevation was markedly lower (0.3±0.21 ng/ml) and infarctions were smaller. cVR was noted in 3 of 4 pigs, accounting for a much lower proportion of rhythm duration (0.424±0.805% over the same period, p=0.028) and cycle length was longer (904±8.09 ms, p=0.0012). All animals survived until the predetermined endpoint and exhibited normal global and regional ventricular function and perfusion.

Experimental animals. All experimental procedures were performed in accordance with the National Institutes of Health guidelines for the care and use of laboratory animals, and under protocols approved by the Institutional Animal Care and Use Committee at the Indiana University School of Medicine. Fourteen, specific pathogen free (Michigan State University) Yorkshire cross domestic pigs (28-38 kg) of mixed gender were used in the study. We enrolled 6 pigs in the control group, while 9 received ASC and 4 received ASC admixed with heparin (ASC-H).

General anesthesia, pre and post-operative treatment Antiplatelet therapy was administered as follows: aspirin 325 mg per oral (PO), 24 hours prior to the cell or vehicle delivery, followed by 81 mg PO daily till euthanasia. Anticoagulation therapy was administered intravenously (IV) as follows: an unfractionated heparin 300 U/kg IV bolus at the beginning of the procedure and the ACT was maintained at more than 300 sec. General anesthesia was induced with an intramuscular injection of TELAZOL® (4.4 mg/kg), xylazine (2.2 mg/kg), ketamine (2.2 mg/kg) and atropine (0.05 mg/kg). After intubation, the animal was ventilated with a closed volume cycle respirator and anesthesia unit to maintain a surgical plane of anesthesia using a general inhalation of Isoflurane (1.5-2.5%) and oxygen (2 L/min). The temperature was monitored and stabilized throughout the procedure. BUPRENEX® (0.01 mg/kg) was administered for analgesia after the completion of the cell or vehicle delivery. No beta-blockers or additional pharmaceuticals were administered.

Subcutaneous fat harvest. Dorsal hump subcutaneous fat tissue was collected using an aseptic, sterile technique. A 4-5 cm skin incision was performed lateral to the midline of the dorsal hump region. Hemostasis was controlled by the use of electrocautery, placing pressure and topical epinephrine when needed. Isolation of the adipose stromal-vascular fraction occurred in the cell culture labs in order to maintain sterility. The animal remained under anesthesia while ASC isolation and preparation proceeded.

Isolation of the adipose stromal-vascular fraction (SVF). Fat was isolated using equipment and reagents from TGI-100 kits (Tissue Genesis, Inc, Honolulu, Hi., USA) following the manufacturer's instructions, with modifications as necessary in order to process the subcutaneous pig fat. Briefly, the fat was finely minced in a sterile container before washing 3 times with the supplied buffer solution. The fat was transferred into 50 ml conical screw cap centrifuge tubes before adding 10 ml of filtered ADIPASE™ solution. The tissue was dissociated by incubation in an Enviro-Genie incubator shaker for 2.5 hours, instead of the manufacturer's recommended digestion time for human fat of 45 minutes. The stromal-vascular pellet was recovered by centrifugation, filtered serially through 100 μm then 40 μm diameter pore size polypropylene filters (FALCON®). Viable cell counts were obtained using a hemacytometer and staining was performed with Trypan blue. Only cells larger than approximately 8 μm in diameter were counted. The cells were resuspended in a 20 ml cell the supplied suspension medium immediately before delivery. In the ASC-H group, the suspension medium contained 50 U heparin/ml. The cells were continuously mixed gently by hand until loaded into the syringe for delivery. A preliminary ex-vivo study showed there were no significant alterations in cell viability or diameter range following test deliveries at the anticipated rate (2 ml/min) through the lumen of the selected balloon catheter into culture media.

Angiographic evaluation. Arterial access was obtained via the femoral artery using a cutdown technique. A vascular sheath was used to cannulate the artery. Subsequently, a left heart catheterization was performed using a 7 F Hockey-stick guiding catheter (Boston Scientific Corp, Natick, Mass., USA) for left coronary cannulation and a 6 F pigtail catheter for ventriculography. Hemodynamic monitoring was performed throughout the procedure. The left coronary angiography was performed in the 30 degree left oblique projection. The left ventriculography (LVG) was performed in the right lateral projection. The frame acquisition rate was 30 frames/sec for all images. Both studies were done at baseline, immediately after the cell or vehicle delivery and on the day of euthanasia. The left anterior descending coronary artery (LAD) was selected to assess the angiographic blood flow, which was determined in terms of the TIMI grade,⁸ and TIMI frame count. The TIMI 3 flow is defined by contrast filling the distal coronary bed completely. The TIMI frame count was measured as the numbers of imageframes required to travel from the ostium to the distal LAD. TIMI frame count and the left ventricular (LV) ejection fraction (EF) were determined by each acquired image on a Phillips BV PULSERA® workstation with INTURIS 2.2® software. To evaluate any regional wall motion abnormalities, the degree of motion was computed for each of the 5 segments provided by the same software.

⁹⁹Tc-sestamibi SPECT Imaging and Evaluation. Gated myocardial ^{99m}Tc-Sestamibi SPECT studies were acquired 7 days after delivery, with a dual-detector camera (Siemens ECAM); a low-energy, high-resolution collimator; a 20% symmetric window at 140 keV; a 64×64 matrix; an elliptic orbit with 32 step-and-shoot acquisitions over 180°; and a 25 sec acquisition time per stop. The acquisition was gated by three-lead ECG at 8 frames per cardiac cycle. The summed non-gated SPECT short-axis, vertical long-axis, and horizontal long-axis slices of the left ventricle were displayed for each subject. In addition, polar maps of perfusion, wall motion, and wall thickening were created (Cedars QGS; Cedars-Sinai Medical Center), and values of left ventricular ejection fraction were calculated. Images were reviewed in blinded fashion by a nuclear radiologist.

ASC or vehicle administration. After the baseline left catheterization, a MAVERICK® 3.5×9 mm, non-inflated, over-the-wire balloon (Boston Scientific Corp, Natick, Mass., USA) was placed into the LAD just distal to the first dominant diagonal branch. After removal of the guidewire, 20 ml of the cell preparation was administered through the lumen of the balloon catheter over either 2 or 10 minutes. Multiple doses were administered in 3 animals using a serial doubling dose escalation from 10 million cells, with each dose infused over 2 minutes with 20 minute intervals between doses, achieving an average total cell dose of 93.3±66.2×10⁶ cells. Ventriculography was performed following each dose, to assess functional consequences of cell delivery. Single doses of either 40 million cells (N=4) or 70 million cells (N=1) were administered to the subsequent animals to mimic anticipated clinical delivery methodology using 10 minute-delivery duration with a 3 cc syringe. In ASC-H group, a 10 minute single dose delivery method was used with a target of 40 million cells (N=4). In the control group, 20 ml of vehicle was administered over 2 minutes.

### Evaluation of Myocardial Infarction.

1. Tetrazolium staining. Triphenyltetrazolium chloride (TTC) staining was performed according to the standard protocol.⁹ Hearts were removed immediately after the animals were euthanized and were transversely sliced into sections of ˜1.5 cm thickness along the short axis. Those sections were incubated in a TTC cocktail at a temperature of 37° C. for 20 minutes, at which time a bright red color was considered to represent non-infarcted myocardium.

2. Microscopic evaluation. Each slice of the left ventricle was subdivided into 8 evenly sized wedge-shaped pieces of approximately equal dimensions. All pieces were fixed overnight in 10% buffered formalin and then transferred to 70% ethanol prior to the tissue processing through paraffin. Five-micron sections were microtomed and placed on positively charged slides. Routine H&E stains were performed and the slides were reviewed by a blinded, board-certified pathologist for evidence of myocardial infarction. When noted, radial and circumferential infarction dimensions were traced to permit estimation of the infarction area, assuming a basal-apical dimension of the slice thickness.

3. Cardiac troponin I. Blood samples were collected at baseline, 24 hours after the cell delivery, and immediately prior to sacrifice. The serum cardiac troponin I (cTn-I) was measured by a GLP lab using chemiluminescent immunoassay method (IMMULITE®, Siemens AG, Munich, Germany), validated in a porcine model.

Evaluation of nerve sprouting. Left ventricular thin section tissues from the territory of distal LAD at the papillary muscle level were evaluating cardiac nerve sprouts by immunocytochemical staining. We included animals in this analysis which were sacrificed on day 7 after delivery. Growth-associated protein 43 (GAP 43) and Tyrosine hydroxylase (TH) staining were performed using a modified immunocytochemical ABC-peroxidase method as described previously.¹⁰ The density of stained nerves was quantified by computerized morphometry and expressed as the total area of positive staining per unit area (μm²/mm²).¹¹

Electrocardiographic monitoring. Unanesthetized and anesthetized electrocardiograms (ECG) were collected using the LIFESHIRT™ system (Samples at 200 Hz, VivoMetrics Inc, Ventura, Calif., USA), a wearable, ambulatory cardiopulmonary monitoring system, which allowed collection of electrocardiographic data continuously via a modified limb lead I. The data was captured on removable flash memory drive and decrypted for analysis in VIVOLOGIC® 3.1 software. ECG monitoring was performed continuously following fat harvest for at least 3 days, and for up to 7 days, until the time of euthanasia. All animals in the ASC group also received 24 hours of continual monitoring at 5 days post-delivery. The baseline ECG in each animal was collected one to two weeks prior to the cell delivery. A cardiac electrophysiologist reviewed all the recordings. To quantify the ventricular rhythm, the ECG recordings were analyzed using a custom-built algorithm that utilized methods similar to those described by previous studies^{12,13}. The algorithm entailed applying a 1^{st} order differentiator to effectively high-pass filter the digital ECG signal, making the QRS complexes discernible from the other interwave components and background noise. Subsequently, cross-correlation was performed between each QRS as defined by differentiation and a user-defined QRS template in order to identify the on- and offset and peak of each QRS complex and to quantitatively determined the similarity between template and all beats in order to identify wide QRS, which were identified as premature ventricular complexes (PVCs). The ECG analysis algorithm was developed and implemented using MATLAB 7.0.1. Prior to use for this study, the algorithm was tested against three records from MIT-BIH Arrhythmia Database: there was less than 5% error between the algorithm-detected rhythm and the database values. Additionally, the algorithm-detected wide QRSs were validated by visual inspection. A significant proarrhythmic event was defined as follows: bradycardia lasting more than 3 seconds except for sinus bradycardia during sleep, accelerated idioventricular rhythm (AIVR), ventricular tachycardia (VT) and ventricular fibrillation during any event. Consecutive ventricular rhythm (cVR) was defined as 3 or more consecutive PVCs, while interruption by 2 or more, narrow, sinus-originated QRS complexes was considered to be a termination of the cVR episode. VT was defined as cVR faster than 100 beats per minute (BPM); AIVR was defined as cVR slower than 100 BPM. The burden of ventricular rhythm was quantified as a percentage of the number of QRS complexes of ventricular origin (wide QRS) per the total number of QRS complexes in a given hour duration, and the characteristics of the cVR were analyzed.

Statistical analysis. All values are presented as mean±standard deviation. For the duration of VT, the median value was also provided. The data were compared with either ANOVA or Student's t-test as appropriate. In case of serial values in the study animal, we used a general linear model with repeated measures analysis using SPSS® 15.0 software (SPSS Inc, Chicago, Ill., USA). The differences between the data were considered significant at a P<0.05.

### Example 1

In brief, adipose stromal cells were harvested from fat for reinjection into the coronary arteries of pig hearts and it was observed that these cells formed macromolecular aggregates. Microscopic inspection of isolated cells in solution indicated that cell aggregates ranged from 4 cells (the lowest number of cells in an aggregate that could be reliably counted) to numbers which were too numerous to count. This aggregation phenomenon was observed with ASCs isolated from porcine and human fat that were either freshly harvested or had been cultured. The inventors conducted experiments to determine the nature of the properties of ASCs that contributed to aggregation, and lead to the discovery of methods and compositions to block ASC aggregation. Accordingly, the inventors demonstrate methods to inhibit or reduce ASC aggregation which are generally useful to those using ASCs, or other cell types with similar aggregative properties, for example, for therapeutic uses and transplantation into a subject.

The inventors demonstrate that the aggregative properties of isolated ASCs are inhibited by treatment with substances that block this process, which prevents the formation of macromolecular aggregates that may block arteries and thereby reduce blood flow to tissues. Accordingly, the inventors demonstrated a method for enhancing the safety profile of ASCs when delivered as a therapeutic agent for treatment of diseases of vascularized tissues and organs.

The inventors demonstrated methods and agents to block aggregation ASCs by combination of the ASC in an admixture with a modulator of ASC aggregation, (e.g. an inhibitor of ASC aggregation) such as, but not limited to ionic agents (e.g., heparin), chelating agents (e.g., EDTA), proteolytic agents.(e.g., trypsin or dispase), intracellularly acting agents that block expression of surface displayed attachment proteins (e.g., integrins), or any other agent that modifies attachment or self-aggregative properties of ASCs.

Characteristics of the study animals. The body weights were 34.4±3.01 kg in the control group, 37.7±4.4 kg in the ASC group and 36.6±1.91 kg in the ASC-H group. Heart weights were 167.7±26.1 g, 171.4±30.3 g and 145.0±16.58 g respectively for the three groups. The proportions of males to females were 2:4, 4:5 and 3:1 in the three groups, respectively. None of these values differed statistically between the groups.

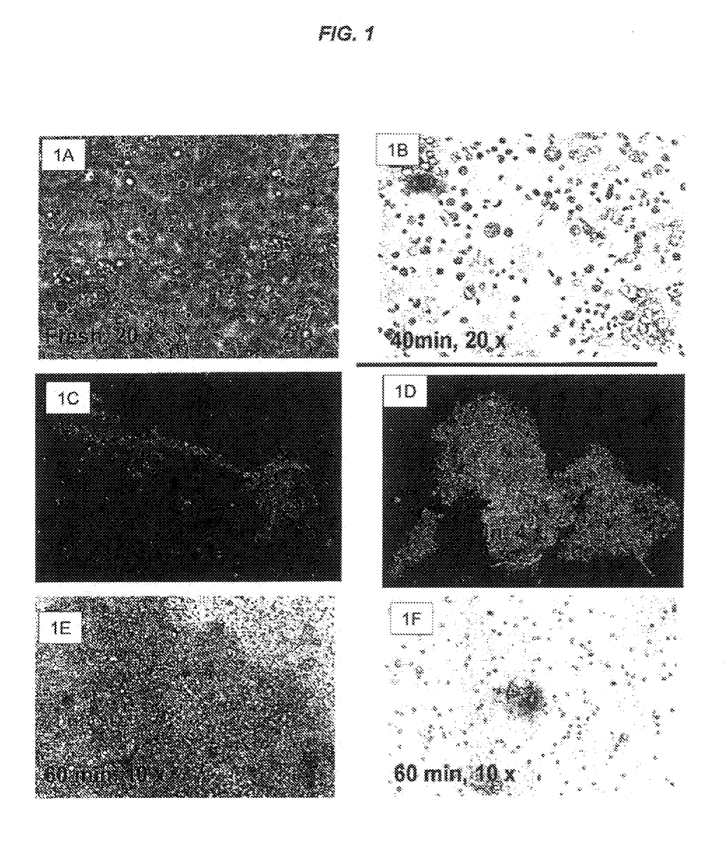

Preparative yield of ASC. The amount of harvested fat was 36.1±15.6 g and the total number of cells obtained was 9.8×10⁷±5.7×10⁷ cells. The cell yield/gram of fat was 2.7×10⁶±1.2×10⁶ cells/g. This yield was achieved by quadrupling the digestion time which was necessitated due to the fibrous nature of subcutaneous porcine fat compared to pliable human fat. The ASC diameter was 9.9±2.5 μm (range of 8-15 μm). Gross observations of freshly isolated ASC showed evidence of cellular aggregation over time (first noted approximately 15 min after re-suspension), even with gentle continuous mixing (FIG. 1). The admixture of ASC with heparin immediately following preparative suspension mitigated this aggregation (data not shown).

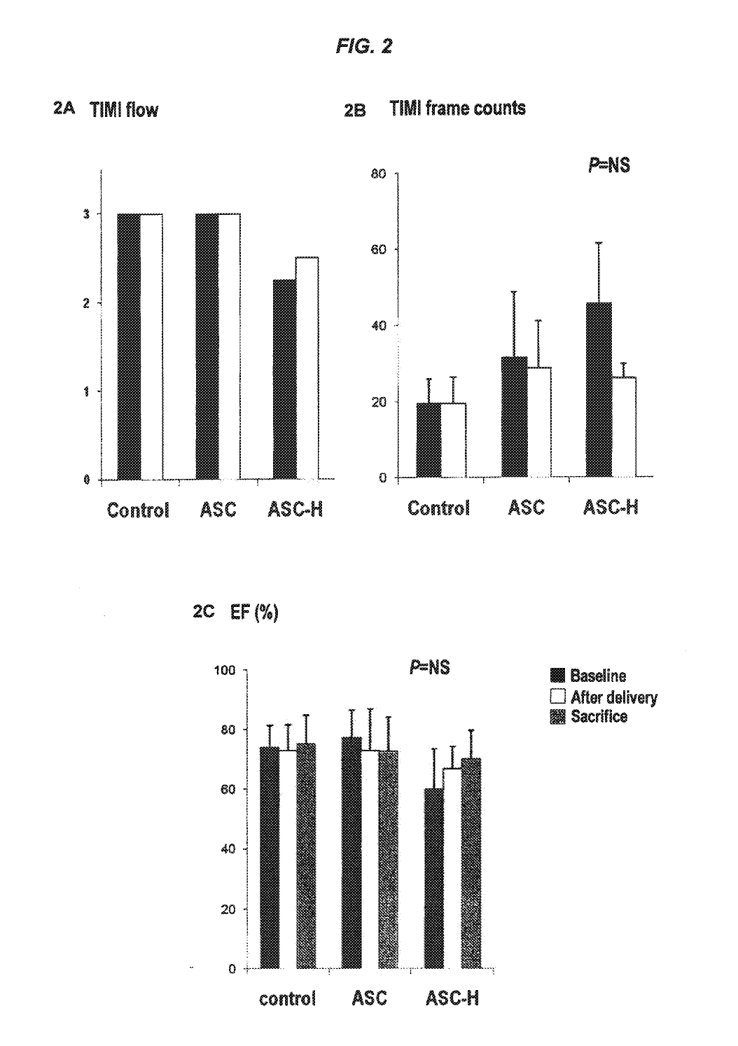

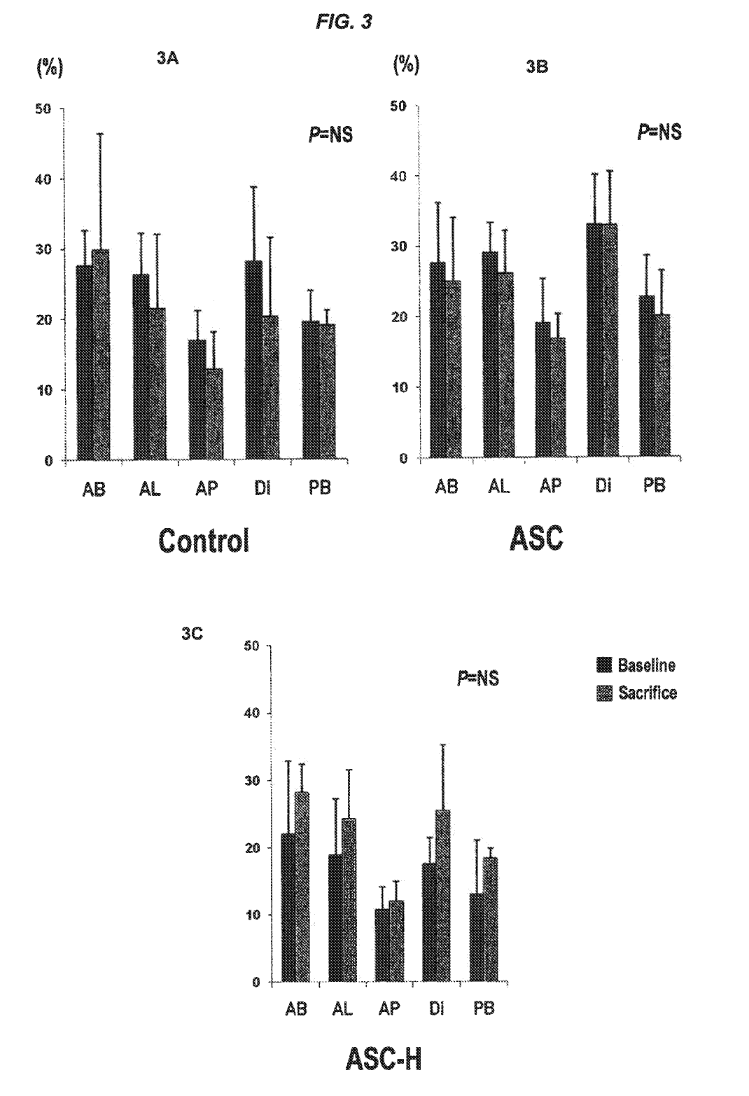

Angiography and Ventriculography. The baseline TIMI flow was 2.25 in the ASC-H group, and 3 in the ASC and control groups. Coronary angiography taken immediately after the ASC, ASC-H or vehicle delivery did not reveal reductions in flow, but rather slight increases in the cell delivery groups. The TIMI framecounts at baseline and immediately after ASC delivery were 31.4±17.3 and 28.6±12.5, respectively. The TIMI framecounts at baseline and immediately after ASC-H delivery were 45.8±15.6 and 26.0±3.8, which is higher than ASC delivery alone or the control group, which had a baseline and post-delivery TIMI framecounts were 19.5±6.3 and 19.5±6.7, respectively. The intra-group framecounts difference was not statistically significant in all groups. Left ventriculography (LVG) revealed baseline left ventricular ejection fraction (LVEF) of 77.3±9.1%, 60.1±13.3% and 74.0±7.46% in the ASC, ASC-H and control groups, respectively. This baseline LVEF was significantly lower in the ASC-H group (P<0.05) compared to the ASC alone or control group (FIG. 2). LVG obtained immediately after cell delivery and at sacrifice did not show any evidence of newly developed regional wall motion abnormalities in comparison with baseline data (FIG. 3). The LVEF values immediately after the delivery and at sacrifice in the 7 day ASC group were 73.0±13.7% and 72.7±11.5%, in ASC-H group were 67.0±7.3%, 70.1±9.6; and in the control group were 73.1±8.4% and 75.3±9.53%, respectively.

⁹⁹Tc-sestamibi SPECT Resting perfusion images were taken at sacrifice (day 7) from 5 of 9 pigs in the ASC group, and did not reveal any deviation from the normal porcine myocardial pattern.

### Example 2

### Myocardial Infarction

### Cardiac Troponin-I

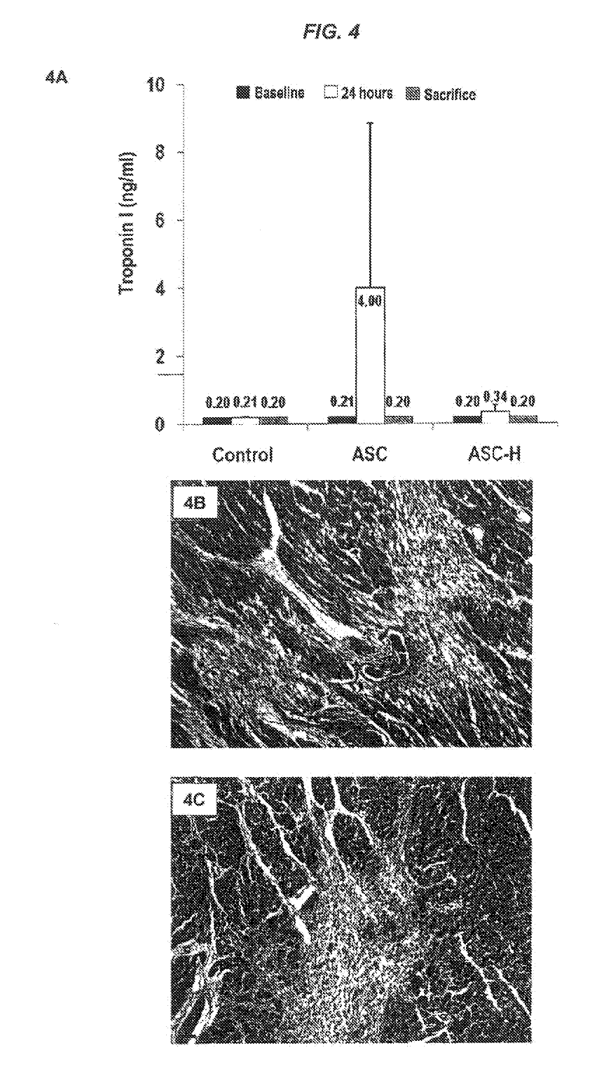

Baseline cTn-I levels in all groups were <0.2 ng/ml (reference level: <0.2 ng/ml). At 24 hours after cell delivery, there was no detectable elevation in the control group. Seven of 9 (about 77%) of the animals in the ASC group (4.0±4.8 ng/ml) and all 4 animals (100%) in ASC-H group (0.3±0.21 ng/ml) exhibited an elevation of the cTn-I level at 24 hours. Thus, the inventors demonstrate at the 24 hour cTn-I timepoint, the ASC-H had a reduction of cTn-I elevation with heparin treatment as compared to ASC group. In all pigs, the cTn-I levels returned to normal 7 days after delivery. (FIG. 4)

### Myocardial Infarction: TTC Staining and Histological Evaluation

In the control group, TTC staining and histological analysis did not show any infarctions. In the ASC group, 3 pigs had scattered small infarctions visible by TTC staining; and histological evaluation revealed myocardial infarctions in 6 of 9 pigs (about 66%). (FIG. 4) Five pigs had microscopic infarctions in the LAD territory and infarctions in remote sites from the cell delivery were noted in 2 pigs. No infarctions spanned more than 2 serial slices. One pig (#8) had very small microinfarctions with a recanalized small coronary artery that was 95% occluded by a partially organized thromboembolus, even though the cTn-I level was not elevated. The occlusion was composed of small clear cells covered with a thin fibrous cap. The coronary arteries of all other pigs were free of thrombi and no occlusions were seen.

In contrast to the ASC group, the inventors surprising demonstrated that in the ASC-H group, TTC staining did not show infarctions. Three pigs had microscopic infarctions in the LAD territory and remote site infarctions were noted in 3 pigs by histological evaluation. Only one pig (25%) exhibited only remote site infarction. Combining the cTn-I and histological results, all of the cell-delivered pigs had myocardial microinfarctions (Table 1). However, the inventors surprisingly discovered that the size of myocardial infarction was markedly diminished in the ASC-H group.

### Myocardial Infarction: Cardiac Nerve Sprouting

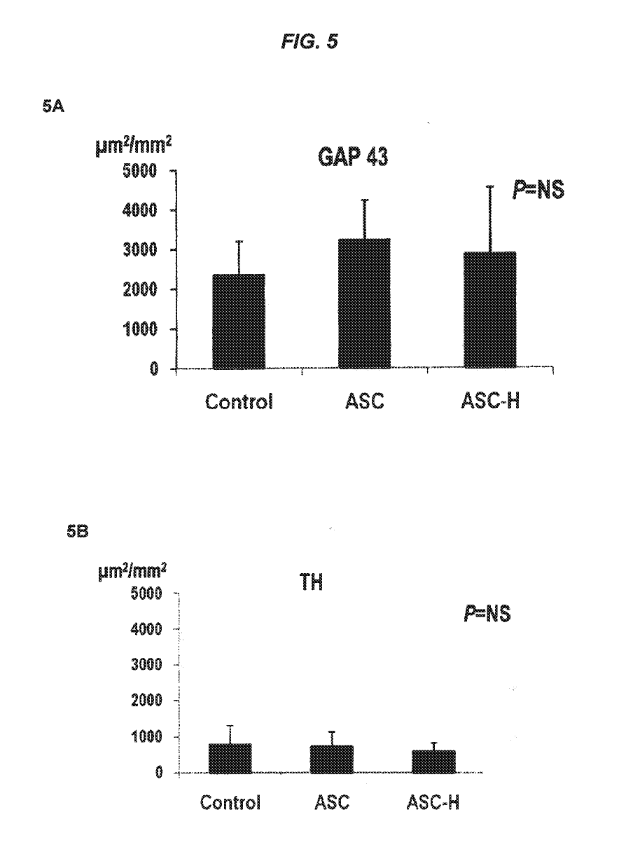

The mean GAP 43⁺ nerve density was 2335.64±863.6 μm²/mm², 3212.94±1016.6 μm²/mm², and 2869.28±1679.9 μm²/mm² in control, ASC, and ASC-H groups respectively (FIG. 5). There were no significant differences between any of the groups. Additionally, there was also no difference in TH staining of the sections from control, ASC or ASC-H groups, which were 779.0±538.1 μm²/mm², 715.15±422.7 μm²/mm², and 589.69±228.5 μm²/mm², respectively.

### Example 3

Arrhythmia: At baseline, no animals exhibited significant arrhythmia. Neither cell nor vehicle delivery caused any acute ventricular arrhythmias, bradycardia or conduction block. In the control group, the continuous recordings of 3 of 6 pigs (50%) were suitable for analysis. These pigs were monitored for 70.5±20.03 (48.9-74.0) hours after vehicle delivery, and revealed no significant dysrrhythmias. In the ASC group, the group of multiple infusion pigs (sacrificed by protocol at 1 day after the cell delivery), was monitored for at least 16 hours following cell delivery, with an average monitoring time of 17.9±1.12 hours. Among this group, one pig had cVR episodes which persisted until the time of euthanasia. The second group (sacrificed at 7^{th} day) of pigs was monitored for 107.8±45.15 hours after the cell delivery. Among the second group of pigs, 4 of 5 (80%) had significant cVR episodes which terminated spontaneously as described below. In the ASC-H group, animals were monitored for 62.9±15.9 hours after the cell delivery. Three of them had significant cVR episodes. There were no animals which died suddenly.

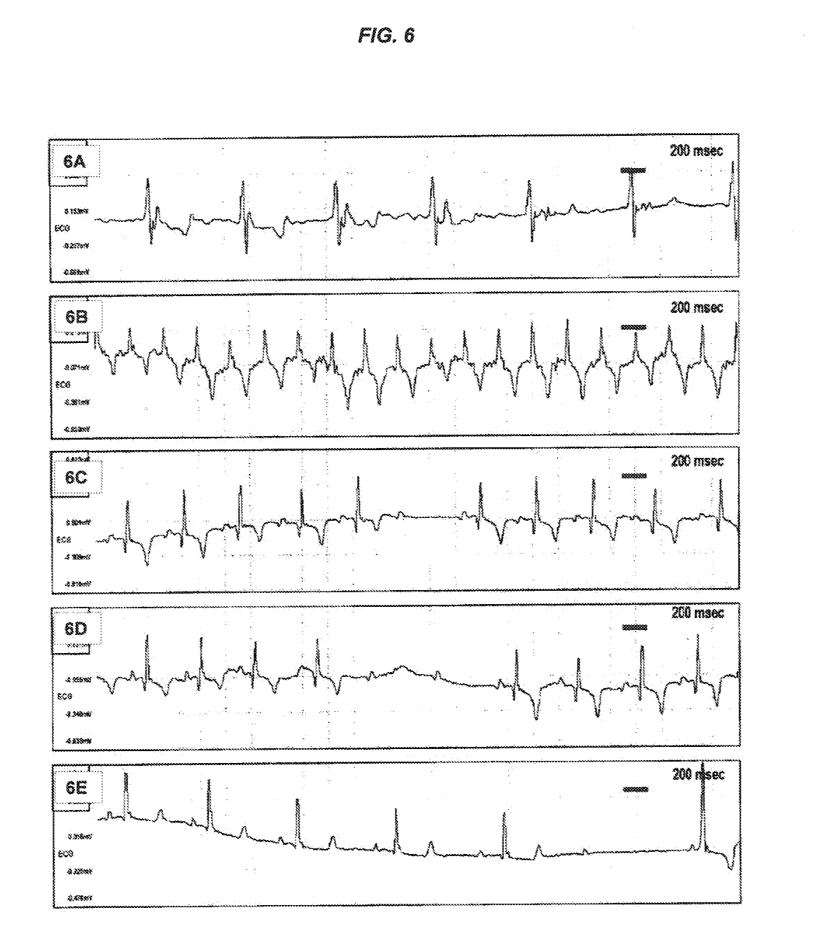

Bradycardia: The recordings from three animals (#6, #7 and #10) showed transient runs of 2:1 atrioventricular (AV) block, 3:1 AV block, and Wenckebach AV block, peaking in frequency approximately 5 hours after the cell delivery. However, those were not significant (FIG. 6).

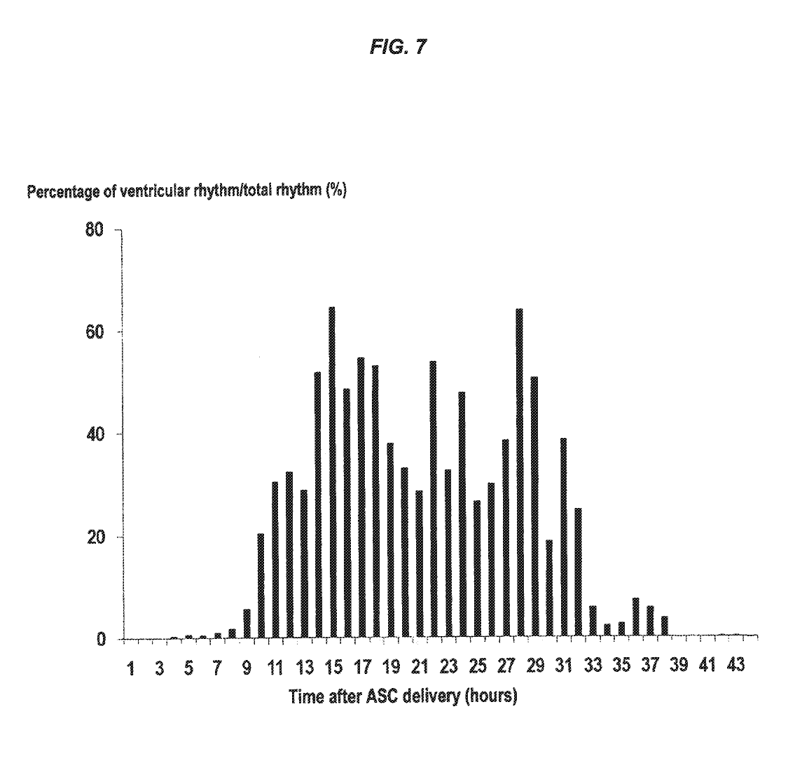

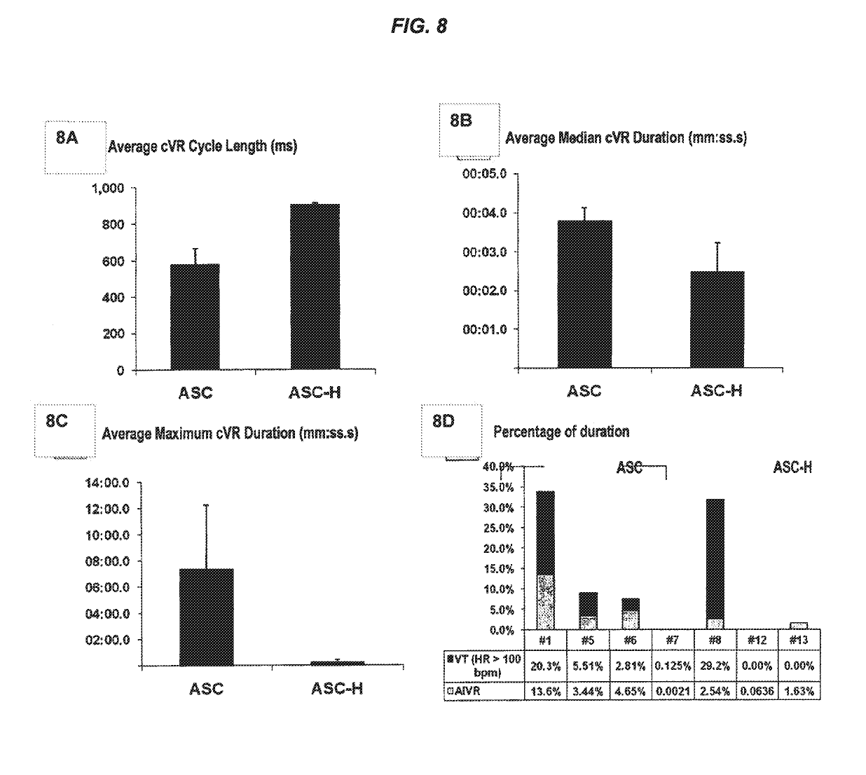

Tachycardia: Onset of cVR was preceded by PVCs; and the duration of non-consecutive ventricular beats was noted as longer than the cVR. In the ASC group, the PVCs emerged 3.5±3.19 hours after the cell delivery and ventricular rhythms were observed to occur within the first 2 days after cell delivery. They were represented as the percentage of a ventricular rhythm per the total QRS complex number (FIG. 7). The mean initiation time of the cVR was 6.4±1.39 hours after the cell delivery. Among the pigs in the ASC group which exhibited cVR, the average time required for the disappearance of the cVR was 28.9±5.64 hours. The average cycle length of the cVR was 586.0±110.8 ms. The longest duration of any cVR episode was 16.28 minutes and the median duration of the cVR episodes was 4.1 s. The incidences of cVR occupied 18.9±13.9% of the analyzed total recording duration. The durations of VT and AIVR were 12.9±10.4% and 6.0±4.9%, respectively. All the pigs in the ASC group were monitored for a complete period of 24 hours on day 5 post-cell delivery, and at that time there were 161.2±349.3 (0˜786) isolated ventricular premature beats per 24 hours (<0.1% of total QRS complexes). In the ASC-H group, the average cycle length of the cVR was 791.1±91.3 ms. The longest duration of any cVR episode was 6.97 minutes and the median duration of the cVR episodes was 2.5 s. The duration of cVR occupied 5.49±8.6% of the analyzed total recording duration. The durations of VT and AIVR were 3.3±5.7% and 2.2±2.9%, respectively. To compare the incidence of cVR in ASC and ASC-H group, the same time duration (21 hours) after cell delivery was analyzed. The duration of cVR in that period was 16.4±15.3% and 0.424±0.805% in ASC and ASC-H group respectively (p=0.029). The cycle length of cVR in ASC group was 577±86 ms. In ASC-H group, the cVR cycle length was longer (904±8.09 ms, p=0.0012) than the ASC group. The inventors demonstrate in the ASC-H group, the incidence of cVR was lower, cVR episode durations were shorter, and average cycle lengths were longer than in the ASC group (FIG. 8) Each animal number can be identified in the table shown in Table 1.

Table 1 shows results of MI and arrhythmia in each animal from 3 groups; control, ASC and ASC plus heparin. Cell: X 10⁶, M: multiple dose, H: Heparinized, TTC: Triphenyltetrazolium chloride staining, MI; myocardial infarction, LAD: left anterior descending coronary artery, Tn I: cardiac troponin-I (ng/ml).

| | | | histologic | histologic | | | |
|---|---|---|---|---|---|---|---|
| pig ID | cell | TTC-MI | MI-LAD | MI-remote | Tn I_24 h | arrhythmia | sacrifice |
| #1 | 150 m | + | + | − | 11.6 | + | 1 day |
| #2 | 143 m | − | − | − | 1.3 | − | 1 day |
| #3 | 70 m | − | − | − | 0.46 | − | 1 day |
| #4 | 10 | − | − | − | 1.4 | − | 1 day |
| #5 | 70 | + | + | − | 11 | + | 7 days |
| #6 | 40 | + | + | + | 8.3 | + | 7 days |
| #7 | 40 | − | + | − | 1.5 | + | 7 days |
| #8 | 40 | − | − | + | 0.2 | + | 7 days |
| #9 | 40 | − | + | − | 0.2 | − | 7 days |
| #10 | 40 H | − | + | − | 0.25 | + | 7 days |
| #11 | 40 H | − | − | + | 0.22 | − | 7 days |
| #12 | 40 H | − | + | + | 0.65 | + | 7 days |
| #13 | 23 H | − | + | + | 0.25 | + | 7 days |
| #14 | vehicle | − | − | − | 0.2 | | 7 days |
| #15 | vehicle | − | − | − | 0.24 | − | 7 days |
| #16 | vehicle | − | − | − | 0.2 | − | 7 days |
| #17 | vehicle | − | − | − | 0.2 | − | 7 days |
| #18 | vehicle | − | − | − | 0.2 | | 7 days |
| #19 | vehicle | − | − | − | 0.2 | | 7 days |

### Example 4

This is the first study to evaluate the effects of delivery of freshly isolated adipose derived stromal cells to normal myocardium in the absence of pre-existing myocardial infarction. In this study, the inventors were able to detect myocardial infarctions accompanied by transient, self-limited post-infarction cVR noted to persist for approximately 40 hours following the ASC delivery. However, the inventors demonstrate that a mixture of ASC preparation with heparin surprisingly mitigates the potential for added myocardial damage due to cell aggregation.

### Microinfarction

The intracoronary route for delivery of stem cells offers the potential advantage of a readily adopted delivery technique with broad cellular distribution and enhanced consistency of retention in comparison with other delivery methods.^{14,15} However, the intracoronary approach raises the potential for concern of adverse consequences of cellular trapping in microvessels, manifested by decreased distal coronary flow⁷ and the possibility of infarction in the event of occlusion of vascular distributions without significant collateral supply. Key variables determining the probability for important microvascular occlusion include cell dimension, dose, concentration, aggregation, and surface molecules determining cell adhesive properties. Previous studies have evaluated infusion of cultured MSCs derived from bone marrow, adipose tissue and umbilical cord; the diameters of these cells have been in the range of 20 μm.¹⁶⁻¹⁸ Studies at cell doses of ˜10×10⁶ and 50×10⁶ have exhibited microinfarctions following intracoronary delivery of cultured MSCS.^{17,18} The study by Valina et al.¹⁶ described the choice of a dose of 2 million cultured MSCs, which did not induce infarctions. Taken together, these studies indicate that the occurrence of microinfarctions upon infusion of MSCs into normal myocardium may be a dose-dependent phenomenon. Accordingly, the inventors investigated if intracoronary infusion and trapping of ASCs might be accompanied by occasional infarction of normal tissue, and demonstrate that it is important to specifically evaluate the functional consequence of infusion of freshly isolated, uncultured ASCs, particularly given the significant size disparity between such cells (diameter ˜10 μm) and expanded MSCs.

Indeed, the inventors demonstrate herein that the infusion of these smaller ASCs into normal myocardium is accompanied by localized infarctions in the region of infusion and, occasionally, in remote areas.

### Cardiac Nerve Sprouting

Cardiac nerve sprouting is a response to myocardial infarction,¹⁹ rapid atrial pacing,²⁰ radiofrequency catheter ablation,²¹ and heart transplantation.²² Pak et al.¹¹ reported cardiac nerve sprouting after direct injection of MSC in myocardial infarction model. It was reported that these treatments increased the magnitude of cardiac nerve sprouting 2 months after injection. Therefore, the inventors evaluated the relation between nerve sprouting and arrhythmia after cell delivery. The inventors demonstrate that there no significant difference in sprouts formed in hearts of the control and cell delivery groups, such ASC or ASC-H treated groups. Thus, the inventors demonstrate that nerve sprouting in response to ASC delivery has no role in arrhythmogenesis.

### Post-Infusion Arrhythmia

Prior studies have reported a transient increase in the frequency of VT episodes occurring following intramyocardial injection of skeletal myoblasts, peaking in the range of 11-30 days after the stem cell delivery,^{23,24} and concerns have been raised that electrically uncoupled skeletal myoblasts may become a substrate for reentrant tachycardias.²⁵ However, these studies have not reported acute arrhythmias in the acute phase after the injection. On the contrary, studies using bone marrow derived MSCs have not reported an increased incidence of arrhythmias in either animals or patients post-myocardial infarction.^{1,26} The study by Vulliet et al. using a normal dog model reported that the intracoronary delivery of bone marrow MSCs caused a transient AIVR at 24 hours after cell delivery, accompanied by microinfarctions.¹⁸ However, little detailed information on the arrhythmia was provided. Fotuhi et al.,²⁷ reported that freshly isolated adipose-derived mononuclear cells were not proarrhythmic in pigs when administered to post-infarction myocardium. During a monitoring period of 43 days, implantable loop recording did not capture sustained arrhythmias. However, in contrast to the invention here, the study by Fotuhi et al., employed administration of magnesium and amiodarone during the MI procedure, which likely served to attenuate the subacute arrhythmogenicity. The inventors discovery differs in two significant aspects: the inventors did not employ antiarrhythmic agents at the time of cell infusion; and the deliveries were conducted into normal myocardium in order to permit a sensitive evaluation on the effects of cell trapping in regions of normal myocardium adjacent to or interlaced with regions of myocardium at risk for infarction. This enabled the inventors to demonstrate localized infarctions and self-limited peri-infarction arrhythmias of ASC delivery that might be anticipated in the event of cell trapping in normal myocardium. It also enabled the inventors to demonstrate that ASC in the presence of Heparin reduced localized infarctions. It is important to note that animals which received ASC cells and manifested VT or AIVR, all demonstrated substantial resolution of the arrhythmia over the typical 48 hour time period associated with clinical resolution of peri-infarction arrhythmias; and none died suddenly.

### Example 5

The inventors have recently determined that administration of autologous porcine adipose stromal vascular fraction (SVF) cells (or adipose-derived stromal cells (ASCs)) directly into normal hearts via intracoronary artery delivery resulted in significant elevation of cardiac damage markers, increased incidence of arrhythmia, and histological evidence of regional ischemia. Visual inspection before administration indicated the occurrence of time-dependent aggregation of cells within SVF, which may have created micro-emboli within the coronary vasculature. Empirical tests with readily available agents demonstrated that aggregation and subsequent microinfractions could be substantially diminished by pre-treating SVF with heparin before infusion. The inventors then demonstrated factors mediating aggregation as well as identified agents suitable for use in humans that block or inhibit the SVF aggregation.

SVF was isolated from either excised dorsal hump (porcine) or lipoaspirated subcutaneous (human) adipose tissues using reagents and protocols supplied by Tissue Genesis Inc. (TGI). Cells were used either fresh or after enrichment for stromal cells, containing adipose-derived stem cells (ADSC), by attachment to and growth on uncoated tissue culture plastic. Low passage (P1-P4) cultured ASCs were detached from plastic by 2 mM EDTA treatment. 2−3×10⁶ fresh SVF or cultured ASCs were incubated for increasing times in 1 ml of TGI suspension buffer, containing human serum proteins, under varying conditions as indicated in Figure legends. Cell suspensions were vigorously rocked during incubation. At the times indicated the suspensions were observed macroscopically, noting aggregate size and number, as well as microscopically, to quantitate aggregates (defined as a tightly associated grouping of 4 or more cells) as well as individual cells.

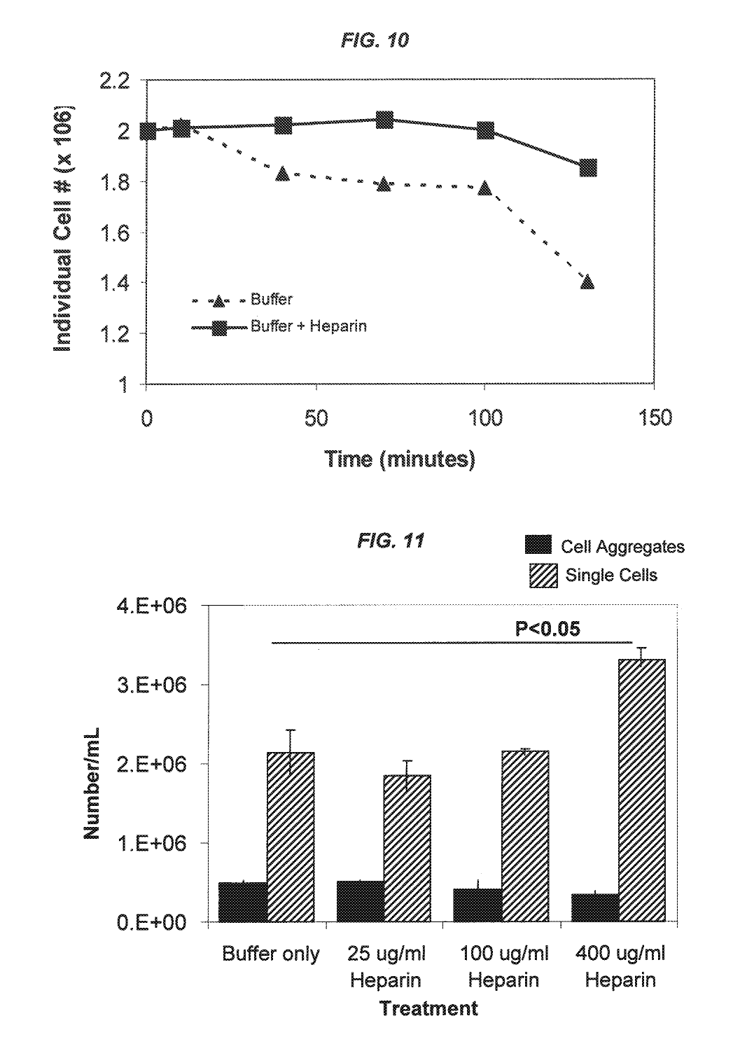

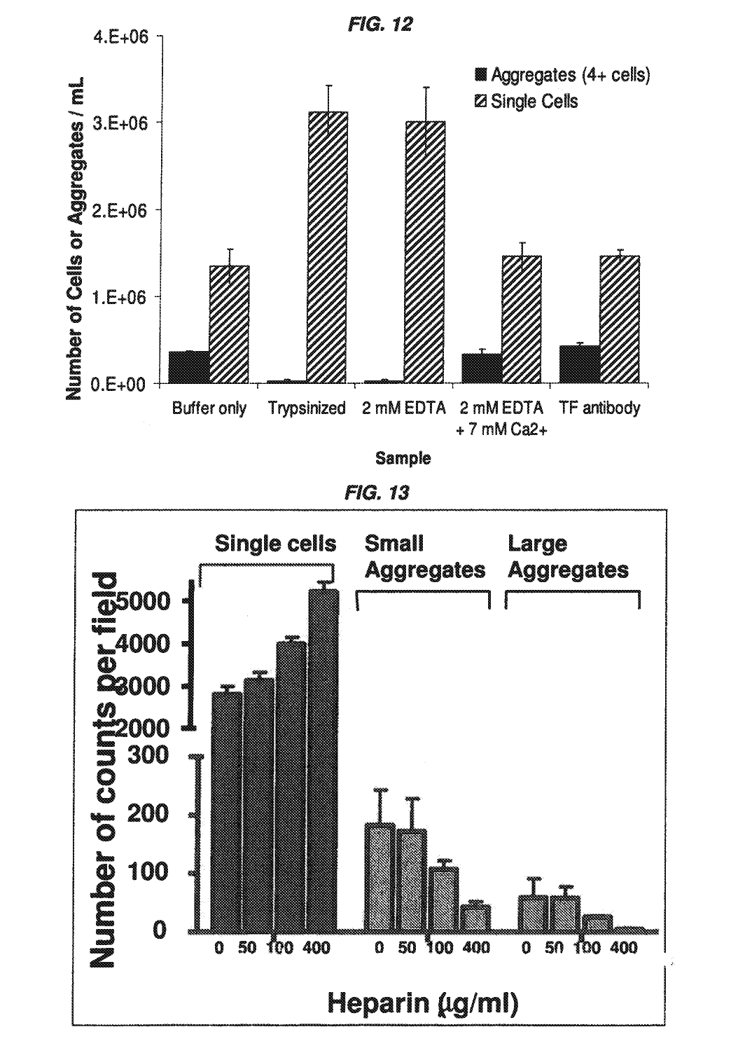

Aggregates of SVF and ASCs formed within 20 minutes of resuspension and, in some cases, obtained macroscopic sizes. (FIG. 9). Aggregation of cells was inhibited by adding heparin (400 μg/ml final conc.) to the buffer, (FIG. 10) Inhibition of ASC aggregation by Heparin was concentration dependent with 400 μg/ml (100 U/ml) being the most effect concentration used in this study. (FIG. 11). Cell aggregation was inhibited by trypsinization and by chelation of divalent cations with EDTA. The effect of EDTA was reversed by the co-addition of Ca²⁺. Aggregation was not affected by inhibition of tissue factor (TF), indicating that it is distinct from coagulation. (FIG. 12). Formation of aggregates of all sizes was blocked by Heparin treatment; however, aggregation still persisted at the highest concentration used (400 μg/ml) (FIG. 13). Even the lowest concentration of Heparin used (50 μg/ml) was inhibitory towards ASC proliferation and the highest concentration (400 μg/ml, totally inhibited proliferation (data not shown))

Thus the inventors demonstrate that ASCs possess aggregative properties that are mediated by proteinaceous surface factors and that require divalent cations but are distinct from classic coagulation pathways. In one embodiment, inhibition of the surface factor(s) and/or cell-surface receptors or proteins responsible for promoting aggregation can be performed, which include proteoglycans and other proteins which mediate adhesion amongst cells as well as between cells and extracellular matrices. The inventors demonstrate heparin and similar agents can inhibit ASC aggregation; thereby, increasing their clinical safety and utility.

### Safety Considerations

The inventors have demonstrated that the occasional cellular aggregates observed in freshly isolated preparations of ASC poses a safety concern with respect to the potential forming emboli in coronaries that may trigger arrythmogenic ischemic events. Here, the inventors demonstrate a way to prevent and reduce cellular aggregates of freshly isolated ASC by administering the ASC in conjunction with Heparin.

The inventors therefore demonstrate a method to inhibit ASC aggregation to avoid myocardial damage using a common ionic anticoagulant heparin was tested. This was not anticipated as heparin is not normally used in this context. In the normal context, Heparin is normally used for inhibition of platelet aggregations. In particular, Heparin is not used in repair, rather, it is used as a blood thinner in subjects with blood clotting tendency.

The inventors demonstrate that the admixture of ASC with heparin immediately following preparative suspension indeed mitigated and significantly reduced ASC aggregation, and also decreased the incidence of arrhythmia and the size of myocardial infarctions.

In summary, despite the limited diameter of freshly isolated ASC compared to other mesenchymal cell-types, the inventors demonstrate that intracoronary delivery of freshly isolated ASC is associated with microinfarctions and temporally limited episodes of ventricular arrhythmia. Suprizingly, the inventors demonstrate that by preparing a heparin-ASC mixture prior to injection, it reduces the incidence (e.g. number or frequency) and severity (e.g. size) of the microinfarctions and ventricular arrthmia. Additionally, the inventors demonstrate it is advantageous to deliver such cells, e.g., an admixture of ASC and heparin to human subject, similar to as was done with pig subjects herein, via intracoronary routes which reduces arrhythmia during the days immediately following the infusion procedure.

### REFERENCES

All references, patents and patent applications cited herein and throughout the specification are herewith incorporated by reference in their entirety.
1. Wollert K C, Meyer G P, Lotz J, Ringes-Lichtenberg S, Lippolt P, Breidenbach C, Fichtner S, Korte T, Hornig B, Messinger D, Arseniev L, Hertenstein B, Ganser A, Drexler H. Intracoronary autologous bone-marrow cell transfer after myocardial infarction: the BOOST randomised controlled clinical trial. Lancet. 2004; 364(9429):141-148.
2. Ghostine S, Carrion C, Souza L C, Richard P, Bruneval P, Vilquin J T, Pouzet B, Schwartz K, Menasche P, Hagege A A. Long-term efficacy of myoblast transplantation on regional structure and function after myocardial infarction. Circulation. 2002; 106(12 Suppl 1): 11 31-136.
3. Kern S, Eichler H, Stoeve J, Kluter H, Bieback K. Comparative analysis of mesenchymal stem cells from bone marrow, umbilical cord blood, or adipose tissue. Stem Cells. 2006; 24(5):1294-1301.
4. Izadpanah R, Trygg C, Patel B, Kriedt C, Dufour J, Gimble J M, Bunnell B A. Biologic properties of mesenchymal stem cells derived from bone marrow and adipose tissue. J Cell Biochem. 2006; 99(5):1285-1297.
5. Rehman J, Traktuev D, Li J, Merfeld-Clauss S, Temm-Grove C J, Bovenkerk J E, Pell C L, Johnstone B H, Considine R V, March K L. Secretion of angiogenic and antiapoptotic factors by human adipose stromal cells. Circulation. 2004; 109(10):1292-1298.
6. Planat-Benard V, Silvestre J S, Cousin B, Andre M, Nibbelink M, Tamarat R, Clergue M, Manneville C, Saillan-Barreau C, Duriez M, Tedgui A, Levy B, Penicaud L, Casteilla L. Plasticity of human adipose lineage cells toward endothelial cells: physiological and therapeutic perspectives. Circulation. 2004; 109(5):656-663.
7. Freyman T, Polin G, Osman H, Crary J, Lu M, Cheng L, Palasis M, Wilensky R L. A quantitative, randomized study evaluating three methods of mesenchymal stem cell delivery following myocardial infarction. European heart journal. 2006; 27(9): 1114-1122.
8. Comparison of invasive and conservative strategies after treatment with intravenous tissue plasminogen activator in acute myocardial infarction. Results of the thrombolysis in myocardial infarction (TIMI) phase II trial. The TIMI Study Group. N Engl J Med. 1989; 320(10):618-627.
9. Fishbein M C, Meerbaum S, Rit J, Lando U, Kanmatsuse K, Mercier J C, Corday E, Ganz W. Early phase acute myocardial infarct size quantification: Validation of the triphenyl tetrazolium chloride tissue enzyme staining technique. American Heart Journal. 1981; 101(5):593-600.
10. Zhou S, Jung B-C, Tan A Y, Trang V Q, Gholmieh G, Han S-W, Lin S-F, Fishbein M C, Chen P-S, Chen L S. Spontaneous stellate ganglion nerve activity and ventricular arrhythmia in a canine model of sudden death. Heart Rhythm. 2008; 5(1):131-139.
11. Pak H-N, Qayyum M, Kim D T, Hamabe A, Miyauchi Y, Lill M C, Frantzen M, Takizawa K, Chen L S, Fishbein M C, Sharifi B G, Chen P-S, Makkar R. Mesenchymal Stem Cell Injection Induces Cardiac Nerve Sprouting and Increased Tenascin Expression in a Swine Model of Myocardial Infarction. Journal of Cardiovascular Electrophysiology. 2003; 14(8):841-848.
12. Lander P, Berbari E J. Principles and signal processing techniques of the high-resolution electrocardiogram. Prog Cardiovasc Dis. 1992; 35(3):169-188.
13. Last T, Nugent C D, Owens F J. Multi-component based cross correlation beat detection in electrocardiogram analysis. Biomed Eng Online. 2004; 3(1):26.
14. Hou D, Youssef E A, Brinton T J, Zhang P, Rogers P, Price E T, Yeung A C, Johnstone B H, Yock P G, March K L. Radiolabeled cell distribution after intramyocardial, intracoronary, and interstitial retrograde coronary venous delivery: implications for current clinical trials. Circulation. 2005; 112(9 Suppl):1150-156.
15. Strauer B E, Brehm M, Zeus T, Kostering M, Hernandez A, Sorg R V, Kogler G, Wernet P. Repair of infarcted myocardium by autologous intracoronary mononuclear bone marrow cell transplantation in humans. Circulation. 2002; 106(15):1913-1918.
16. Valina C, Pinkernell K, Song Y H, Bai X, Sadat S, Campeau R J, Le Jemtel T H, Alt E. Intracoronary administration of autologous adipose tissue-derived stem cells improves left ventricular function, perfusion, and remodelling after acute myocardial infarction. European heart journal. 2007; 28(21):2667-2677.
17. Moelker A D, Baks T, Wever K M, Spitskovsky D, Wielopolski P A, van Beusekom H M, van Geuns R J, Wnendt S, Duncker D J, van der Giessen W J. Intracoronary delivery of umbilical cord blood derived unrestricted somatic stem cells is not suitable to improve LV function after myocardial infarction in swine. Journal of molecular and cellular cardiology. 2007; 42(4):735-745.
18. Vulliet P R, Greeley M, Halloran S M, MacDonald K A, Kittleson M D. Intra-coronary arterial injection of mesenchymal stromal cells and microinfarction in dogs. Lancet. 2004; 363(9411):783-784.
19. Cao J M, Chen L S, KenKnight B H, Ohara T, Lee M H, Tsai J, Lai W W, Karagueuzian H S, Wolf P L, Fishbein M C, Chen P S. Nerve sprouting and sudden cardiac death. Circ Res. 2000; 86(7):816-821.
20. Akira H, Chang C-M, Zhou S, Chou C-C, Yi J, Miyauchi Y, Okuyama Y, Fishbein M C, Karagueuzian HS, Chen P H D, Lan S, Chen P-S. Induction of Atrial Fibrillation and Nerve Sprouting by Prolonged Left Atrial Pacing in Dogs. Pacing and Clinical Electrophysiology. 2003; 26(12):2247-2252.
21. Okuyama Y, Pak H-N, Miyauchi Y, Liu Y-B, Chou C-C, Hayashi H, Fu K J, Kerwin W F, Kar S, Hata C, Karagueuzian H S, Fishbein M C, Chen P-S, Chen L S. Nerve sprouting induced by radiofrequency catheter ablation in dogs. Heart Rhythm. 2004; 1(6): 712-717.
22. Kim D T, Luthringer D J, Lai A C, Suh G, Czer L, Chen L S, Chen P-S, Fishbein M C. Sympathetic nerve sprouting after orthotopic heart transplantation. The Journal of Heart and Lung Transplantation. 2004; 23(12):1349-1358.
23. Menasche P, Hagege A A, Vilquin J T, Desnos M, Abergel E, Pouzet B, Bel A, Sarateanu S, Scorsin M, Schwartz K, Bruneval P, Benbunan M, Marolleau J P, Duboc D. Autologous skeletal myoblast transplantation for severe postinfarction left ventricular dysfunction. Journal of the American College of Cardiology. 2003; 41(7):1078-1083.
24. Dib N, McCarthy P, Campbell A, Yeager M, Pagani F D, Wright S, MacLellan W R, Fonarow G, Eisen H J, Michler R E, Binkley P, Buchele D, Korn R, Ghazoul M, Dinsmore J, Opie S R, Diethrich E. Feasibility and safety of autologous myoblast transplantation in patients with ischemic cardiomyopathy. Cell Transplant. 2005; 14(1):11-19.
25. Fouts K, Fernandes B, Mal N, Liu J, Laurita K R. Electrophysiological consequence of skeletal myoblast transplantation in normal and infarcted canine myocardium. Heart Rhythm. 2006; 3(4):452-461.
26. Mills W R, Mal N, Kiedrowski M J, Unger R, Forudi F, Popovic Z B, Penn M S, Laurita K R. Stem cell therapy enhances electrical viability in myocardial infarction. Journal of molecular and cellular cardiology. 2007; 42(2):304-314.
27. Fotuhi P, Song Y-H, Alt E. Electrophysiological consequence of adipose-derived stem cell transplantation in infarcted porcine myocardium. Europace. 2007; 9(12):1218-1221.
28. Ehlert F A, Goldberger J J. Cellular and pathophysiological mechanisms of ventricular arrhythmias in acute ischemia and infarction. Pacing Clin Electrophysiol. 1997; 20(4 Pt 1):966-975.
29. Friedman P L, Stewart J R, Fenoglio J J, Jr., Wit A L. Survival of subendocardial Purkinje fibers after extensive myocardial infarction in dogs. Circ Res. 1973; 33 (5): 597-611.

1. An admixture comprising a combination of an isolated population of adipose-derived stromal cells (ASCs) and an inhibitor of ASC aggregation.

2. (canceled)

3. The admixture of claim 1, wherein the inhibitor of ASC aggregation is an ionic agent or a chelating agent or a proteolytic agent.

4. The admixture of claim 1, wherein the inhibitor of ASC aggregation is selected from the group consisting of: heparin, EDTA, trypsin or dispase or an analogue thereof.

5. (canceled)

6. (canceled)

7. (canceled)

8. (canceled)

9. (canceled)

10. The admixture of claim 1, wherein the inhibitor of ASC aggregation is an agent which blocks the expression of integrin.

11. The admixture of claim 1, wherein the ASC are human or harvested from a human subject.

12. (canceled)

13. The admixture of claim 1, wherein the ASCs are selected from the group selected from freshly isolated ASC, cultured ASC, genetically modified ASC.

14. (canceled)

15. (canceled)

16. The admixture of claim 1, wherein the ASC in combination with a modulator of ASC
is cryopreserved.

17. A method of inhibiting formation of macromolecular aggregates of adipose-derived stromal cells (ASC), comprising contacting a population of ASC with an inhibitor of ASC aggregation.

18. The method of claim 17, wherein the macromolecular aggregate of ASC comprises at least 4 adipose-derived stromal cells.

19. (canceled)

20. The method of claim 17, wherein the inhibitor of ASC aggregation is an ionic agent, or a chelating agent or a proteolytic agent.

21. The method of claim 17, wherein the inhibitor of ASC is selected from the group consisting of heparin, EDTA, trypsin or disease or an analogue thereof.

22. (canceled)

23. (canceled)

24. (canceled)

25. (canceled)

26. (canceled)

27. The method of claim 17, wherein the inhibitor of ASC aggregation is an agent which blocks the expression of integrin.

28. The method of claim 17, wherein the ASC are human or harvested from a human subject.

29. (canceled)

30. The method of claim 17, wherein the ASCs are selected from the group consisting of freshly isolated ASC, cultured ASC, or genetically modified ASC.

31. (canceled)

32. (canceled)

33. (canceled)

34. Use of the admixture of claim 1 for administering to a subject in need of a transplant of adipose-derived stromal cells (ASC).

35. The use of the admixture of claim 34, wherein the subject is suffering with insufficient cardiac function or disease or disorder.

36. The use of the admixture of claim 35, wherein the disease or disorder is heart failure or myocardial infarction.

37. (canceled)

38. (canceled)

39. (canceled)

40. The use of the admixture of claim 34, wherein the subject is human.

41. The use of the admixture of claim 40, wherein the ASC in the admixture are harvested from the same subject to which the admixture is administered to.

42. A use of heparin for inhibiting aggregation of freshly isolated ASC.

43. A method of reducing the consequences of a myocardial infarction in a subject, the method comprising the steps of
a. freshly isolating ASC from the subject;
b. forming a mixture of the freshly isolated ASC and heparin; and
c. delivering via intracoronary catheter the mixture to post-infarction myocardium of the subject.
wherein the delivery of the mixture of freshly isolated ASC and heparin reduce the consequences of a myocardial infarction in the subject.
